(19) United States
(12) Patent Application Publication
(10) Pub. No.: US 2011/0257368 A1
(43) Pub. Date: Oct. 20, 2011

(54) PURIFICATION OF RECOMBINANTLY PRODUCED INTERFERON

(75) Inventors: Xiaoyu Yang, Basking Ridge, NJ (US); Gary J. Vellekamp, Highland Park, NJ (US)

(73) Assignee: Schering Corporation, Kenilworth, NJ (US)

(21) Appl. No.: 13/141,269

(22) PCT Filed: Dec. 17, 2009
(86) PCT No.: PCT/US09/68433

§ 371 Date: Jun. 21, 2011

Related U.S. Application Data
(60) Provisional application No. 61/140,361, filed on Dec. 23, 2008.

(51) Int. Cl. C07K 1/18

(52) U.S. Cl. 530/351

(57) ABSTRACT

The present invention provides a method for separating desired interferon isoforms from undesired interferon isoforms that involves subjecting the isoforms to anion exchange column chromatography and a biphasic elution procedure. A strong elution solution is used in the first elution phase to facilitate elution of the desired isoform from the column and a weak elution solution is used in the second phase to suppress elution of the desired isoforms.

### FIELD OF THE INVENTION

The present invention relates to the purification of interferon produced by recombinant organisms. In particular, the present invention relates to the chromatographic separation of desired interferon isoforms, e.g., isoforms that have a desired secondary disulfide bond structure and lack chemical adducts, from undesired interferon isoforms produced by such organisms.

### BACKGROUND OF THE INVENTION

Interferons are cytokines that exhibit antiviral, antiproliferative and immunomodulatory activities. Because of such activities, different types of interferons have been approved for treating diseases such as hepatitis, various cancers and multiple sclerosis.

The interferons (IFNs) may be divided into three main groups based on their biological and physical properties. In humans, Type I IFNs consist of five classes:

alpha (IFN-α), beta (IFN-(β) epsilon (IFN-ε), kappa (IFN-K), and omega (IFN-ω). Interferon gamma (IFN-y) is the only known Type II interferon. Type III includes IFN-lambdas. See, e.g., Antonelli, G., New Microbiol. 31:305-318 (2008).

Multiple subtypes of IFN-a proteins are expressed in humans and many other species, with 12 different mature subtypes identified in humans (Bekisz, J. et al., Growth Factors 22(4):243-251 (2004); Antonelli, G., supra; Pestka, S. et al., Immunol. Reviews 202:8-32 (2004); Diaz, M.O., et al., J. Interferon Cytokine Res 16:179-180 (1996)). Human IFN-a subtypes share 75-99% amino acid sequence identity and a mature sequence of 166 a.a. except for IFN-a2, which has 165 a.a. due to a deletion at position 44. Also, some IFN-a subtypes exist in variant forms, such as IFN-α2 which has at least 3 allelic forms: IFN-a2a, IFN-a2b, and IFN-a2c.

The most conserved feature shared by Type I IFNs is the disulfide bond: 2 disulfide bonds are present in IFN-a and IFN-w, while one is present in IFN-13. The disulfide bonds in IFN-a are between Cys1-Cys 99(98) and Cys29-Cys139(138), with the residue numbers in parenthesis referring to IFN-a2. The single disulfide bond in IFN-β, is between Cys31-Cys141. The IFN-a Cys29-Cys139(138) and IFN-β Cys31-Cys141 bonds are apparently critical in binding of these IFNs to the Type I IFN receptor complex and thus in maintenance of their biological activities (Bekisz et al., supra).

While IFNs may be obtained from their natural sources, recombinant techniques permit the production of large quantities of these proteins from non-natural sources, such as bacteria and other microorganisms that have been transformed with a DNA molecule encoding the desired IFN protein. The production of IFNs by recombinant organisms typically includes a multi-step purification process, including chromatography on various media, to remove contaminants originating from the host organism or culture media as well as structural isoforms of the IFN protein intended to be produced. See, e.g., U.S. Pat. Nos. U.S. 4765903, US 5196323; European Patent Numbers EP 108585, EP 110302; EP 118808 and EP 0679718; Staehelin et al., J. Biol. Chem 256:9750-9754 (1981); and Secher et al., Nature 285:446-450 (1980).

For example, non-purified and partially purified recombinant IFN-a preparations frequently contain a mixture of structural isoforms of the IFN-a subtype to be produced. Structural isoforms may be divided into three main classes: (1) disulfide bond isoforms, (2) chemical adjunct isoforms and (3) mixed isoforms, which have an altered disulfide bond structure as well as one or more chemical adjuncts. Disulfide bond isoforms include: an oxidized IFN-a monomeric isoform, which has each of the Cys1-Cys 99(98) and Cys29-Cys139(138) disulfide bonds; partially and fully reduced monomeric IFN-a isoforms that lack one or both of these disulfide bonds, respectively; fragments of IFN-α monomers; and IFN-α oligomers, i.e., dimers, trimers and tetramers formed by intermolecular disulfide bonds. Chemical adjunct isoforms, which contain one or more chemical groups attached to the IFN-a amino acid chain, include: a pyruvate-adjunct IFN-a isoform, in which the alpha- amino group of the N-terminal amino acid residue of the IFN-a protein is condensed with the carbonyl group of pyruvate; and a methionine adjunct isoform (International Patent Application publication WO 00/29440; U.S. Pat. No. 5,196,323). Examples of structural isoforms of recombinant IFN-α-2b are shown in FIG. 1.

For therapeutic interferon compositions, the presence of significant amounts of isoforms other than the oxidized monomeric IFN isoform is typically undesired due to concerns that such isoforms may negatively affect the therapeutic or immunogenic properties of the interferon composition. Various techniques have been described for converting undesired structural isoforms into the desired isoform. For example, U.S. Pat. No. 4,432,895 refers to converting oligomeric interferons into monomers using a redox reagent, and WO 00/29440 describes the sequential cleavage of pyruvate groups from chemical adjunct isoforms and oxidation of reduced sulfhydryl groups to disulfide bonds. However, while such isoform conversion techniques typically improve the yield of the desired IFN isoform, significant amounts of undesired structural isoforms may still be present, even if anion exchange chromatography is performed after the conversion step. Thus, a need exists to improve the separation of the desired native isoform, e.g., oxidized, from undesired isoforms in recombinant IFN preparations. The present invention addresses this need.

### SUMMARY OF THE INVENTION

The present invention is based on the inventors' surprising discovery that subjecting a substantially purified recombinant IFN-α-2b preparation to diethylaminoethyl anion exchange (DEAE) chromatography using a novel biphasic elution procedure rather than the standard single phase elution procedure allows efficient separation of the desired, oxidized IFN-α-2b monomer (Isoform 1 in FIG. 1) from undesired isoforms, in particular the pyruvate-adjunct, fully reduced monomer (Isoform 4 in FIG. 1). The first elution phase facilitates elution of the desired IFN-α-2b isoform while the second elution phase suppresses elution of the undesired isoforms. This biphasic elution procedure results in a highly purified IFN-α-2b preparation in a low eluate volume and high yield of the desired isoform. The inventors herein expect that this novel chromatographic procedure can also be used to efficiently separate structural isoforms of other IFN-a subtypes and other IFNs.

Thus, in one aspect, the present invention provides a method of separating an oxidized monomeric isoform of an IFN from undesired isoforms of that IFN in a mixture of recombinantly produced isoforms of the IFN.

The method comprises providing the IFN mixture in a first buffer solution and a chromatography column that is greater than 15 cm in length and packed with an anion exchange resin that is equilibrated with the first or second buffer solution.

The buffered IFN solution is loaded onto the column and then the loaded column is washed with a wash solution.

Next, the first elution phase is performed by applying from 1 to 10 bed volumes of a strong elution solution to the washed column. The strong elution solution is buffered with a first phosphate concentration of 10 to 30 mM and has a pH of between 5.4 and 6.6.

The second elution phase is then performed by applying to the column 2 to 20 bed volumes of a weak elution solution. The elution solution is buffered with a second phosphate concentration that is less than the phosphate concentration in the strong elution solution.

To obtain the highly purified desired IFN isoform, a plurality of eluate fractions containing the desired isoform is collected. Optionally, the eluate fractions in which the amount of the undesired interferon isoforms is less than a desired purity criteria are combined.

In one preferred embodiment, the present invention provides a method of separating IFN-α2b isoform 1 from IFN-α2b isoform 4 in a mixture of recombinantly produced isoforms of IFN-α2b.

The IFN-α2b solution is loaded onto an equilibrated DEAE chromatography column in a loading buffer that consists essentially of 10 mM Tris, 40 mM NaCI, and has a pH of from 7.5 to 8.0. The DEAE column is at least about 20 cm in length and equilibrated with a buffer solution which consists essentially of 10 mM Tris and a pH of 8.0. The loading flow rate is 2 cm per minute.

The loaded column is washed with 3 bed volumes of a wash solution at a flow rate of 2 cm per minute. The wash solution consists essentially of 10 mM Tris and 13 mM NaCI, and has a pH of 8.0.

Next, 6 bed volumes of a strong elution solution are applied to the column at a flow rate of 1 cm per minute followed by a weak elution solution at a flow rate of 1 cm per minute. The strong elution solution consists essentially of 17.5 mM sodium phosphate at pH 5.85 and the weak elution solution consists essentially of 5 mM sodium phosphate at pH 5.85.

Fractions of column eluate containing IFN-α2b isoform 1 are collected and optionally only those collected fractions in which IFN-a2b isoform 4 is less than a desired purity criteria are combined.

### BRIEF DESCRIPTION OF THE DRAWINGS

FIG. 1 illustrates the four structural isoforms typically found in recombinant IFN-α-2b preparations.

FIG. 2 illustrates the results of performing the standard, single phase elution of IFN-a2b isoforms from a DEAE Sepharose Fast Flow column (1cm x 23 cm) using 20 mM sodium phosphate, 20 mM NaCI, pH 6 as elution buffer, with FIG. 2A showing the pH gradient that forms during the elution step, FIG. 2B showing the elution profiles for isoform 1 (IFN-α) and isoform 4 (ISO4) and FIG. 2C showing the purity of individual fractions.

FIG. 3 shows the pH profiles obtained during standard DEAE chromatography using loading buffer only (Control) or a substantially purified IFN-α-2b preparation (IFN) in the same loading buffer as feeds.

FIG. 4 illustrates the effects of the internal pH gradient on IFN-α-2b elution from a DEAE Sepharose Fast Flow column using the specified elution conditions, with the graphs on the left showing overlays of the A280 absorbance, conductivity and pH profiles and the graphs on the right showing the resolution of isoform 1 and isoform 4 as determined by RP-HPLC assay.

FIG. 5 illustrates the characterization of material that was not eluted during standard DEAE chromatography of a substantially purified IFN-α-2b preparation, with FIG. 5A and FIG. 5B showing the analysis of fractions stripped from the column by RP-HPLC and SDS-PAGE, respectively.

FIG. 6 illustrates the absorbance at OD320 and OD320/280 of uneluted material that was stripped from the column and exposed to the indicated pH.

FIG. 7 illustrates DEAE chromatography of an IFNα-2b preparation on a 0.5 cm X 20 cm column, with FIG. 7A showing the pH gradient and absorbance profile,

FIG. 7B showing the elution profiles for isoforms 1 and 4, and Fig. C showing the percentage of isoform 1 and 4 in various eluate fractions.

FIG. 8 shows absorbance, pH and conductivity profiles obtained by eluting IFNa-2b from a DEAE chromatography column in a pH 6.0 elution buffer containing 20 mM sodium phosphate/20 mM NaCI (20/20), 10 mM sodium phosphate/20 mM NaCl (10/20) or 5 mM sodium phosphate/20 mM NaCI (5/20).

FIG. 9 illustrates the impact of elution buffer concentration on DEAE chromatography of IFNa-2b.

FIG. 10 illustrates the impact of salt concentration on the absorbance, pH and conductivity profiles generated by eluting IFNα-2b from a DEAE chromatography column in a pH 6.0 elution buffer containing 20 mM sodium phosphate and a salt concentration of 20 mM NaCI (20/20), 10 mM NaCI (20/10), 5 mM mM NaCI (20/5) or in the absence of NaCl (20/0).

FIG. 11 illustrates the impact of different ionic strengths generated by different salt concentrations on elution of IFNa-2b isoforms 1 and 4 from a DEAE chromatography column.

FIG. 12 illustrates the effect of NaCI on elution of IFNα-2b isoforms 1 and 4 elution in a 10 mM sodium phosphate buffer at pH 6.0.

FIG. 13 illustrates the effect of buffer and salt concentration on separation of IFNα-2b isoforms 1 and 4.

FIG. 14 illustrates the pH effects on IFNa-2b elution using a standard single phase elution process.

FIGS. 15 -27 are described in the Examples.

### DETAILED DESCRIPTION OF THE INVENTION

I. Definitions.

So that the invention may be more readily understood, certain terms are specifically defined below. Unless specifically defined below or elsewhere in this document, all other terms used herein, in particular scientific and technical terms, have the meaning that would be commonly understood by one of ordinary skill in the art to which this invention belongs when used in contexts similar to those used herein.

As used herein, including the appended claims, the singular forms of words such as “a,” “an,” and “the,” include their corresponding plural references unless the context clearly dictates otherwise.

“About” when used to modify a numerically defined parameter, e.g., flow rate, pH, sodium phosphate concentration, means that the parameter may vary by as much as 10% above or below the stated numerical value. Thus, e.g., the term “a flow rate of about 2 cm/min” means that the flow rate may have any value between 1.8 cm/m and 2.2 cm/min. Similarly, the term “about 10 mM Tris” means the Tris concentration may have any value between 9.9 mM and 10.1 mM.

“Consists essentially of” and variations such as “consist essentially of” or “consisting essentially of” as used throughout the specification and claims, indicate the inclusion of any recited elements or group of elements, and the optional inclusion of other elements, of similar or different nature than the recited elements, which do not materially change the basic properties of the specified composition or item. As a nonlimiting example, a weak elution solution consisting essentially of 5 mM sodium phosphate and pH of 5.85 may, e.g., also contain minor amounts of other agents, e.g., potassium phosphate, which do not materially affect the pH, buffering capacity or other properties of the elution solution with respect to separation of the desired IFN isoform from undesired isoforms.

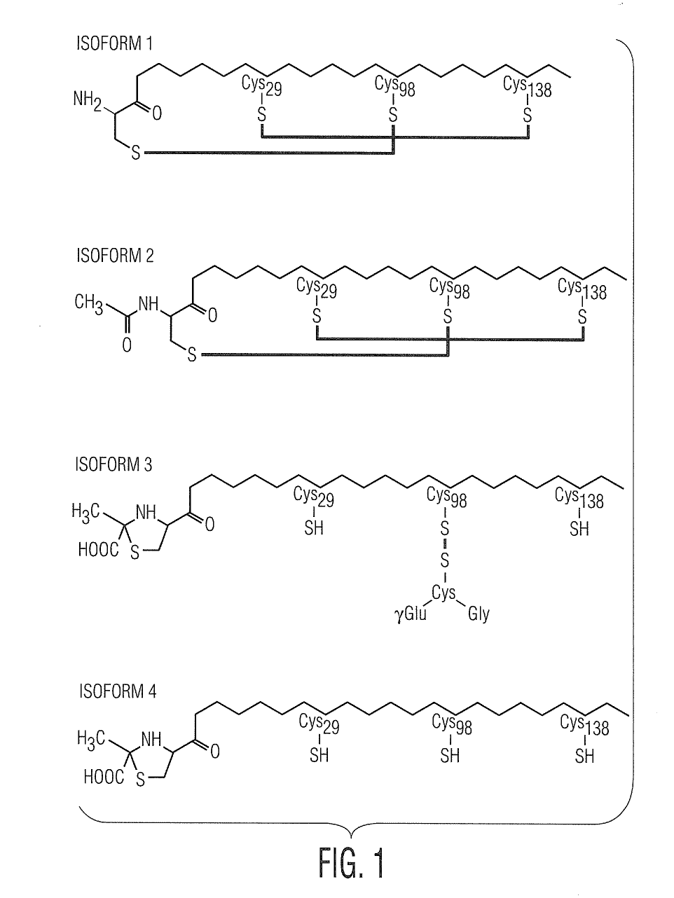

“Iso1” or “ISO1” means IFN-a2b isoform 1 shown in FIG. 1.

“Iso4” or “ISO4” means IFN-a2b isoform 4 shown in FIG. 1.

“NaPi” means sodium phosphate.

“Oxidized IFN monomeric isoform” means a single polypeptide chain that has the natural disulfide bond structure for the subject IFN and has no chemical adjuncts on any amino acids in the polypeptide chain. By natural disulfide bond structure is meant that the number and location of Cys-Cys bonds in the isoform are the same as in the naturally-expressed interferon. Thus, e.g., an oxidized IFN-13 monomeric isoform has a single disulfide bond between Cys31-Cys141, while an oxidized IFN-α2a monomeric isoform has a Cys1-Cys99 bond and a Cys29-Cys139 bond.

“Reduced IFN monomeric isoform” means a single polypeptide chain that has less than the native number of disulfide bonds for the subject IFN and has no chemical adjuncts on any amino acids in the polypeptide chain. A reduced IFN monomeric isoform may be partially or fully reduced depending on the number of naturally occurring disulfide bonds. Thus, e.g., a partially reduced IFN-α2b monomeric isoform lacks either the Cys1-Cys98 bond or the Cys29-Cys138 bond, while a fully reduced IFN-α2b isoform lacks both of these bonds.

“Mixed IFN monomeric isoform” means a single polypeptide chain that has less than the native number of disulfide bonds for the subject IFN and has at least one amino acid in the polypeptide chain modified with a chemical adjunct.

II. Description of Preferred Embodiments

The present invention provides a chromatographic process for separating desired IFN isoforms, e.g., oxidized monomeric isoforms, from undesired IFN isoforms, e.g., reduced IFN isoforms, in recombinant IFN preparations. The process is suitable for the purification of a desired isoform from any recombinantly produced IFN that is naturally expressed by any human or non-human animal species, including any Type I, Type II or Type III IFN, or chimeric or mutant forms thereof in which sequence modifications have been introduced, for example to enhance stability or activity, such as consensus interferons as described in U.S. Pat. Nos. 5,541,293, 4,897,471 and 4,695,629, and hybrid interferons containing combinations of different subtype sequences as described in U.S. Pat. Nos. 4,414,150, 4,456,748 and 4,678,751.

Preferably, the inventive process is used to separate desired and undesired isoforms from recombinantly produced Type I IFNs. Particularly preferred Type I IFNs are recombinantly produced IFN-a proteins, including any of the naturally occurring subtypes IFN-α1, IFN-α2, IFN-α4, IFN-α5, IFN-α6, IFN-α7, IFN-α8, IFN-α10, IFN-α13, IFN-α14, IFN-α16, IFN-α17, IFN-α21, allelic variants of any of these subtypes, or any consensus IFN-a protein in which the amino acid sequence has been designed by selecting at each position the amino acid that most commonly occurs at that position in the various native IFN-a subtypes. More preferably, the recombinantly produced IFN-a protein employed in the present invention is an IFN-α2 (2a, 2b or 2c) and most preferably it is IFN-α2b. The host organism used to express the recombinant IFN may be a prokaryote or eukaryote, e.g., E.coli, B. subtilis or Saccharomyces cerevisiae, preferably E.coli. The conditions of cultivation for the various host organisms are well known to those skilled in the art and are described in detail, e.g., in the textbooks of Maniatis et al. (“Molecular Cloning”, Cold Spring Harbor Laboratory, 1982) and Sambrook et al. (“Molecular Cloning-A Laboratory Manual”, 2nd. ed., Cold Spring Harbor Laboratory, 1989). In particular, the recombinant production of human IFN-a2 proteins is described in Pestka, S. Arch Biochem Biophys. 221:1-37 (1983); International Patent Application publication WO 2004/039996; U.S. Pat. Nos. U.S. 5661009, U.S. 5541293, U.S. 4897471, U.S. 4765903 and U.S. 4530901; and European Patent Application publication EP 032,134.

The recombinant IFN protein may be extracted from the recombinant host or the culture media using any of a variety of procedures known in the art. For example, suitable methods for extracting IFN-a from microorganisms are described in U.S. Pat. Nos. U.S. 4315852, U.S. 4364863, and U.S. 5196323; European Patent Number EP 0679718; and WO 2004/039996.

After extraction, the recombinant IFN preparation, which comprises a mixture of structural isoforms, is preferably subjected to a set of purification steps to produce a substantially pure IFN preparation, which means the preparation is substantially free of non-IFN proteins and other contaminants such as cell debris and nucleic acids, but may contain up to 20% of undesired structural IFN isoforms. Many purification schemes known in the art are suitable for this purpose. Such schemes typically include tandem chromatography on two or more different types of resin, as described in U.S. 5196323, U.S. 4765903, EP 0679718, and WO 2004/039996.

The substantially pure IFN protein preparation obtained from such tandem chromatography typically comprises a buffered solution. Depending on the composition of this solution, as well as the IFN concentration therein, it may be necessary to prepare this IFN solution for loading onto the anion exchange column used in the present invention by adjusting the solution to comprise the components of a loading buffer appropriate for anion exchange chromatography. This adjustment may be performed by techniques known in the art such as dialysis or ultrafiltration.

The loading buffer may be any biologically compatible buffer that does not impact binding of the IFN protein to the anion exchange resin. For example, the loading buffer may contain from 0 to 100 mM of salts such as NaCI, KCI, and sodium acetate. A preferred loading buffer consists essentially of about 10 mM Tris, 0 to 40 mM NaCl, and has a pH of 7.0 to 8.5. A particularly preferred loading buffer consists of 10 mM Tris, 40 mM NaCl and has a pH of 8.0.

The concentration of the IFN in the loading buffer may vary substantially. Typically, the volume of the loading buffer is adjusted to achieve a convenient loading volume, e.g., from 0.5 to 1 bed volumes of the chromatography column, and avoid precipitation of the IFN. In some embodiments, an IFN concentration of between about 1 and about 5 mg/ml is employed. For an IFN-a2 preparation, a preferred concentration of iosoform 1 is about 3.5 mg/ml. The various isoforms present in recombinant IFN preparations may be identified and quantified using techniques known in the art, e.g., such as the RP-HPLC and pyruvate assays described in WO 00/29440. As resin for anion exchange chromatography, a diethylaminoethyl anion exchange (DEAE) media is preferred, with a particularly preferred resin being DEAE with Q-Sepharose Fast Flow (FF) (DEAE Sepharose™FF) from GE Healthcare (Uppsala Sweden or Piscataway, N.J. USA). Other weak anion exchange resins having properties substantially similar to DEAE may also be used.

The anion exchange resin is packed into a chromatography column that is greater than 15 cm in length. In one preferred embodiment, the column is at least 20 cm in length. Other column sizes suitable for use in the present invention include a 1 x 29 cm column.

Prior to the loading of the IFN solution, the anion exchange column is equilibrated with an aqueous buffer suitable for the anion exchange resin and the IFN. The equilibration buffer may be the same or different than the loading buffer. In one preferred embodiment, the column is equilibrated with a buffer consisting essentially of 10 mM Tris, 0- 40 mM NaCI and a pH of 7.0 to 8.5. In another preferred embodiment, the equilibration buffer consists of 10 mM Tris, pH 8.0. If the column has been previously used, it is conveniently regenerated and equilibrated by sequential washings with: 3 bed volumes (B.V.) of 0.5 N NaOH/1 M NaCI; 5 B.V. of H₂O; 3 B.V. of 0.1 N HCI; 6 B.V. of 0.2 M Tris, pH 8.0 and 15 B.V. of 10 mM Tris, pH 8.0, all using a flow rate of 5 cm/ml.

After the buffered IFN solution is loaded onto the column, a wash solution is applied to the column to remove unbound contaminants. The wash solution contains a biologically compatible buffer that does not impact binding of the IFN to the column. For example, in some embodiments, the wash buffer has the same composition as the column equilibration buffer or loading buffer, e.g., consisting essentially of 10 mM Tris, 0 - 40 mM NaCl and a pH of 7.0 to 8.5. In a preferred embodiment, the wash solution consists of 10 mM Tris, 13 mM NaCI and has a pH of 8.0.

The desired isoform is then eluted from the column using a biphasic elution system. This biphasic system is established by applying sequentially to the column two buffered solutions: a strong elution solution, e.g., with a high ionic strength and/or low pH, followed by a weak elution buffer, e.g., having a lower ionic strength and/or higher pH than the strong elution solution.

Conveniently, these elution solutions are buffered with a phosphate, but other biologically compatible buffers may be used. The phosphate concentration in the strong elution solution is from 10 mM to 30 mM and lower in the weak elution solution, e.g., 2.5 to 7.5 mM. The differential in phosphate concentration in the strong and weak elution solutions will vary depending on whether other agents that affect ionic strength, e.g., NaCI or other salts, are present in one or both elution solutions, as well as the pH of each elution solution. For example, the phosphate differential may be somewhat less if the strong elution solution has a lower pH than the weak elution solution. Typically, each of the strong and weak elution solutions will have an acidic pH, preferably in the range of 5.4 to 6.6. The skilled artisan may readily test various combinations of buffer, salt and pH for each of the strong and weak elution solutions to obtain a biophasic system to use for a particular type of IFN.

For preparing phosphate buffered elution solutions, one or both of monosodium phosphate and disodium phosphate may be used and the pH adjusted with a suitable acid or base such as HCI or NaOH. Other phosphate salts such as potassium phosphate may be used in addition to or instead of sodium phosphate. In a preferred embodiment, the biphasic elution is performed using a strong elution solution consisting essentially of 17.5 mM sodium phosphate, pH 5.85 and a weak elution solution consisting essentially of 5 mM sodium phosphate, pH 5.85.

The amount of strong elution solution to use before switching to the weak solution is typically from 1 to 10 bed volumes (B.V.), but may be emperically determined for any particular IFN and set of elution solutions. A test chromatography is performed using the chosen strong elution solution only and the eluate fractions are monitored for the presence of IFN. One less bed volume than the number of bed volumes of strong elution solution that are required to elute the first IFN-containing fraction in monophasic elution would typically be the maximum volume of the strong solution used in the biophasic system. In one embodiment, between 4 and 8 B.V. of strong elution solution are applied. In a more preferred embodiment, the first elution phase is employed with about 6 B.V. of strong elution solution.

The second elution phase may be performed using 2 to 20 B.V. of the weak solution, depending on how much is required to elute a sufficient yield of the desired isoform. For example, in some cases, it may be desired to collect a reduced or chemical adjunct IFN isoform to study its properties. In one preferred embodiment, about 15 B.V. of the weak solution is applied to the column after 6 B.V. of the strong elution solution.

All the above solutions and buffers are typically applied to the anion exchange column at flow rates of 0.2 to 10 cm/minute. The flow rate used will depend upon several factors, such as the equipment to perform the chromatography, the type of anion exchange resin, size of the column, the protein concentration in the IFN solution, and the composition of the elution solutions. Suitable flow rates for each step may be readily determined by the skilled artisan. In some embodiments, the loading buffer, wash solution and elution solutions are applied at flow rates of 1 to 5 cm/min. In other embodiments, the flow rate is from 0.5 to 2.5 cm. Preferably, a flow rate of 2 cm/min is used to apply the loading buffer and wash solution while each of the elution solutions is applied at a flow rate of 1 cm/min.

### Examples

The following examples are provided to more clearly describe the present invention and should not be construed to limit the scope of the invention.

The inventors performed a series of experiments to investigate the mechanisms involved in separation of recombinantly produced IFN-α2b isoforms using DEAE-Sepharose Fast Flow chromatography. Various elution conditions including buffer concentrations, salt concentration, and pH were tested to elucidate the interaction of IFN-α2b isoforms with DEAE Sepharose. Two distinct mechanisms were found to be involved in separation of isoforms 1 and 4.

The first mechanism is consistent with the principle of chromatofocusing, which is characterized by formation of a pH gradient within the column, and is widely used to purify various proteins, particularly closely related isoforms, according to their isoelectric points. In DEAE chromatography for IFN-α2b purification using the standard procedure described below, an internal pH gradient is generated along the length of the column during elution mainly as a result of the interaction of buffer species and DEAE resin. The inventors discovered that isoforms 1 and 4 are eluted at different pHs along the pH gradient. The resolution of isoform 1 and isoform 4 can be improved by using lower concentrations of phosphate elution buffer to reduce the pH gradient slope, consistent with the relationship among buffer concentration, pH gradient slope and column resolution in conventional chromatofocusing.

The second mechanism involves the selective binding of isoform 4 to the column during elution of isoform 1. The inventors discovered that isoform 4 binds to the column more tightly at lower buffer concentrations in the absence of salts, and thus could be effectively separated from isoform 1 under these conditions. However, the elution efficiency for isoform 1 was reduced at lower buffer concentrations; thus, the volume of pooled fractions containing isoform 1 was invariably larger than desired for an efficient commercial protein production process.

Thus, the inventors carried out a second series of experiments to see if they could identify conditions that would allow a robust and efficient elution of IFN-α2b isoform 1 while suppressing elution of IFN-α2b isoform 4.

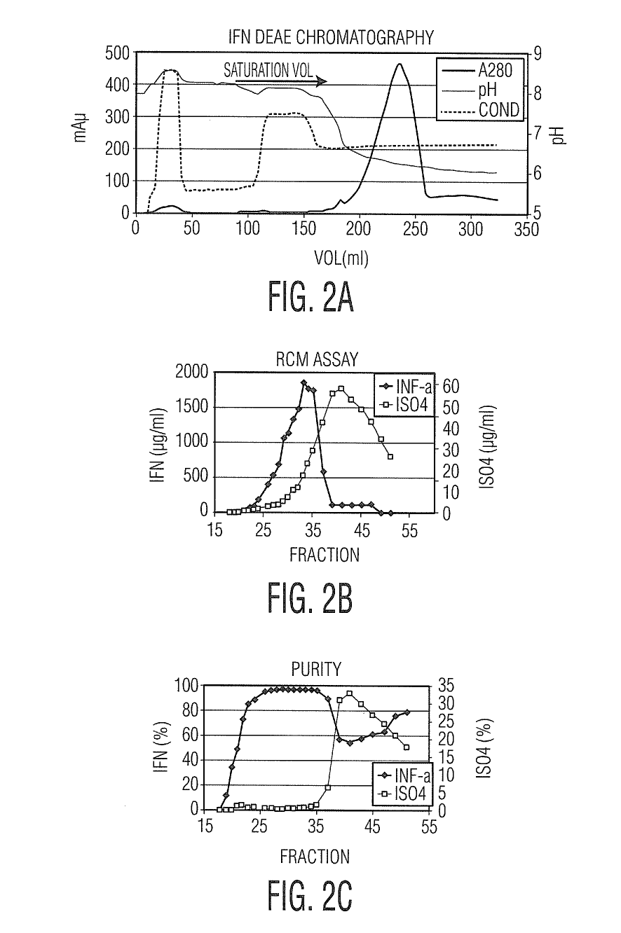

These two series of experiments are described in more detail below, with the results shown in FIGS. 2-27. In these figures, the terms IFN, IFN-α or IFN-alpha refer to IFN-α2b isoform 1 as shown in FIG. 1, and the terms iso4 and 1504 refer to IFN-a2b isoform 4 as shown in FIG. 1.

I. Experimental Procedures

### DEAE Sepharose™ Fast Flow Chromatography.

The AKTAexplorer™ (GE Healthcare, Uppsala Sweden or Piscataway, NJ USA) was used for running DEAE chromatography at 4° C. A 0.5x20 cm column (3.9 ml) or 1 X 29 cm column (23 ml) was packed with DEAE Sepharose^{TM} FF resin (GE Healthcare). Enough resin was poured into the column and allowed to settle under gravity to form an initial bed height of approximately 1 cm above the desired height. The column was regenerated and equilibrated with 3 bed volumes (B.V.) of 0.5 N NaOH/1 M NaCl, 5 B.V. of H20, 3 B.V. of 0.1 N HC1, 6 B.V. of 0.2 M Tris, pH 8.0, and 15 B.V. of 10 mM Tris, pH 8.0 at a flow rate of 5 cm/ml.

### Recombinant IFN-a2b Preparation

We obtained IFN preparations that had been produced in recombinant E. coli, and substantially purified by a series of purification steps. The IFN preparations were provided in a loading buffer containing 10 mM Tris, 40 mM NaCI, pH 7.5-8.0.

### Standard Chromatography Procedure

The IFN solution was injected onto the column equilibrated with 10 mM Tris, pH 8 at a flow rate of 0.4 ml/min for the 3.9 ml column or 0.8 ml for the 23 ml column. The volume of IFN solution loaded was 75% of the column B.V. Three B.V. of wash buffer (10 mM Tris, 13 mM NaCI, pH 8.0) were used to remove unbound materials from the column at the same flow rate. Finally, 9 B.V. of 20/20 elution buffer (20 mM sodium phosphate, 20 mM NaCI, pH 6.0) was employed at a flow rate of 0.2 ml/min to start the elution step. Fractions were collected at 20% B.V. per fraction (0.8 ml). lsoforms 1 and 4 were analyzed by RP-HPLC. Resolution of the isoforms was calculated by dividing the distance of the two isoform peaks by the sum of the peak widths at half peak height.

### Test Chromatography Procedures

In order to examine the behavior of IFN elution, DEAF chromatography was performed using the standard procedure but different elution buffers as described in the Results and Discussion section. The flow rates were identical to ones in the traditional procedure unless otherwise noted.

### II. Results and Discussion—Experimental Series I

A. Formation of an internal pH gradient during IFN DEAE chromatography

The UV absorbance, conductivity, and pH during the standard DEAE chromatography procedure were monitored and shown in FIG. 2. Three UV peaks were observed, bacterial proteins as an early small peak, IFN-a2b isoform 1 as a major middle peak, followed by IFN-a2b isoform 4 and other contaminants as a late broad, minor peak.

Interestingly, the pH of the effluent was relatively constant during the first approximately 3.2 bed volumes of elution before it started to develop internally a non-linear gradient (pH 6 to 7) along the column. The effluent reached the final pH 6 only after 14.2 bed volumes of the elution buffer passed through the column. The pH gradient was likely generated as a result of the interaction between the elution buffer and the DEAE moiety on the sepharose resin. The elution volume before the initiation of the pH gradient is referred to as saturation volume, which is illustrated by an arrow in FIG. 2A. The saturation volume could be related to buffer strength and pH as well as the type of resin.

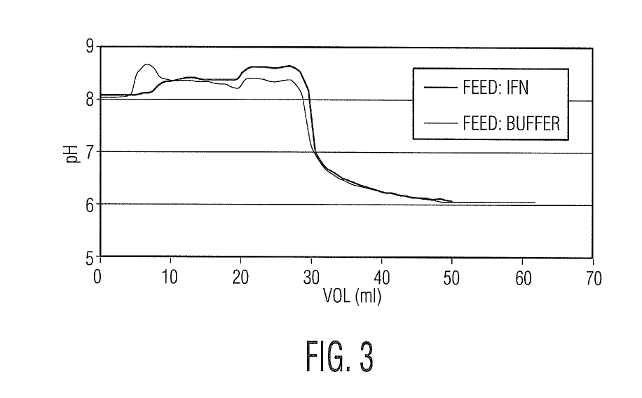

IFN did not appear to contribute to formation of the internal pH gradient as a similar pH profile was observed on “blank” runs, in which loading buffer only was applied to the column (FIG. 3). This result indicated that the driving force for the pH gradient formation was dominated by the interaction between the elution buffer species and the DEAE moiety, while IFN played no significant role. Unlike the pH, the effluent conductivity started to change right after approximately one bed volume of elution but was relatively constant after an initial decline (FIG. 2A).

These data suggest that the pH gradient formation is critical for the proper elution of IFN-a2b isoform 1. The quantitative analysis of IFN-a2b isoforms 1 and 4 by HPLC assay indicated that they were not completely resolved (FIGS. 2B and 2C). Isoform 1 and isoform 4 were eluted at approximately pH 6.3 and 6.1, respectively.

B. The impact of pH gradients on IFN elution

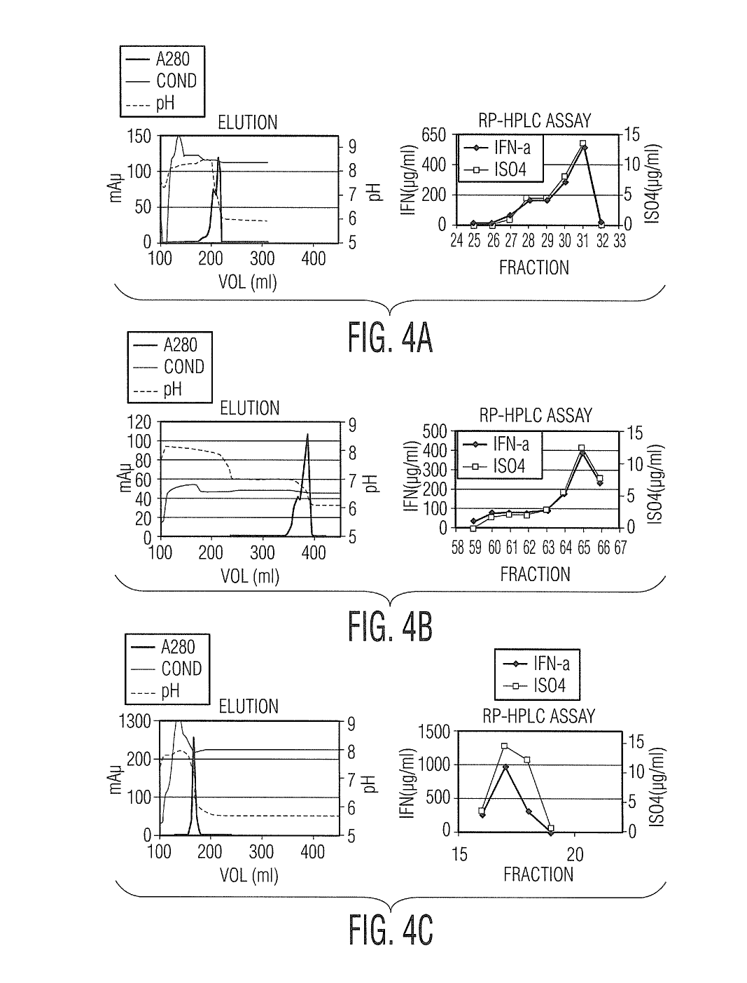

To assess the importance of the internal pH gradient on IFN-a2b isoform resolution, chromatographies were run under various elution conditions without changing other parameters. The use of a pH 6.0 elution buffer containing 10 mM sodium phosphate, 10 mM citric acid, and 20 mM NaCI resulted in a pH gradient having a rather sharp S-shape profile and IFN-a2b eluted as two sharp peaks (FIG. 4A, left graph).

When elution was performed using a pH 6.0 buffer with reduced citrate and phosphate concentrations (5 mM sodium phosphate, 2.5 mM citric acid, 20 mM NaCI), the pH declined more slowly, taking almost twice as much elution volume to reach the pH of the elution buffer (pH 6.0) (FIG. 4B, left panel). Moreover, the pH profile appeared as a two-step cascade as opposed to the one-step observed in the standard procedure where a smoother and shallower concave gradient was seen. The peak IFN did not elute until the pH dropped to approximately 6.2 within the later part of the gradient (FIG. 4B, left panel).

Little to no resolution of isoforms 1 and 4 was observed with either of these citrate/phosphate elution buffers (FIGS. 3A and 3B, right panels), as the pH gradient slope was abruptly increased within the region of pH 6.4 to 6.0 (FIG. 4A and 4B, left panels).

Elution with a 40 mM phosphate buffer (40 mM sodium phosphate, 20 mM NaCI, pH 6) produced a sharp IFN peak (FIG. 4C, left panel). However, isoforms 1 and 4 were poorly resolved (FIG. 4C, right panel), and the pH gradient was noticeably sharper than that in standard runs, even though both the test elution buffer and standard 20/20 buffer had a pH 6.0.

These data suggest that the proper pH gradient is critical for effective separation of IFN-a2b isoforms 1 and 4, and that the slope of the pH gradient can greatly influence the resolution of the column. A sharper pH gradient facilitated elution of both isoforms, but with poor resolution.

C. Chromatofocusing of 1FN on DEAE Sepharose column

As discussed above, we discovered that an internal pH gradient was generated within the DEAE Sepharose column during the elution step and that this pH gradient was critical for the column performance. Therefore, we hypothesized that chromatographic separation of IFN isoforms on DEAE Sepharose chromatography worked by a principle consistent with a unique chromatographic technique called chromatofocusing, which is used for purification of many proteins, particularly those of closely related isozymes of varying isoelectric points (see, e.g., Hutchens TW, 1989 Chromatofocusing in Protein Purification: Principles, High Resolution Methods, and Applications, Janson JC and Ryden L. eds, VCH Publishers, NY, p. 149-174; Giri L. Chromatofocusing in Methods in Enzymology, Deutscher MP. Eds, Academic Press, San Diego, vol 182, p. 380-392).

In typical chromatofocusing procedures, a weak anion-exchange column is equilibrated with a high-pH buffer that facilitates binding of negatively charged proteins. A low-pH buffer that normally employs a commercially available polymeric ampholyte buffer is then introduced to generate an internal linear pH gradient within the column. The proteins will be eluted from the column according to their isoelectric points. When proteins reach to a pH in the column that is equivalent to its pl, the charge of the protein will be net-zero and thus lose its binding affinity to the column and start to elute. As it migrates down the column, the protein front will rebind to the column as pH of the column increases above its pl. Meanwhile, the protein at the backside of the sample zone continues to flow down the column to catch up to the protein front, as it is still uncharged or positively charged. As a result, the protein is focused. The process will be repeated continuously until the protein finally elutes from the column.

Unlike the polyampholyte buffer used in conventional chromatography, the elution buffer in IFN DEAE chromatography consists of only phosphate, which contains three different ionization conjugates of pKa 12.3, 7.3, and 2. However, our data demonstrated that the simple phosphate buffer in the standard procedure was able to form a near-linear or concave pH gradient within pH 6-7, the range between the elution buffer pH and the second phosphate pKa (7.3). IFN was eluted within this narrow range of the pH gradient.

D. Selective binding of IFN-a2b isoform 4

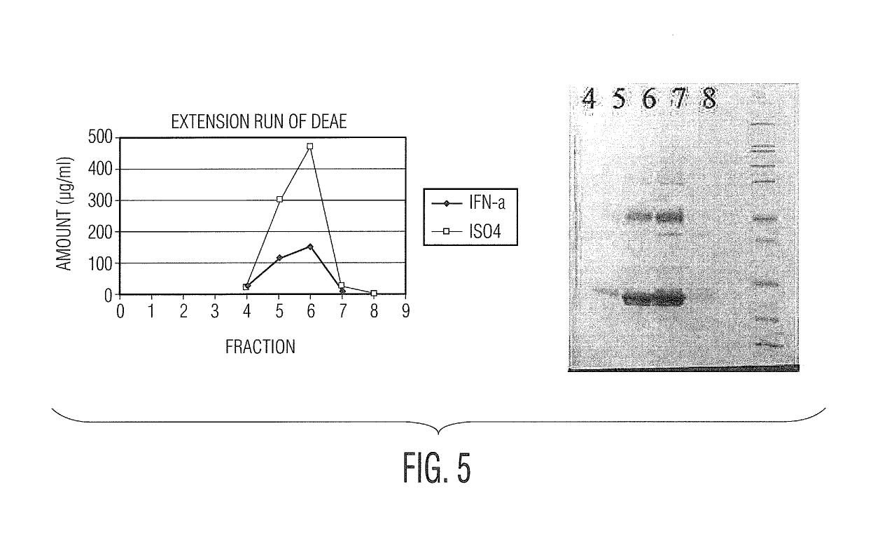

As indicated by mass balance results, there was a large proportion of isoform 4 and other contaminants still binding to the column following the elution step in the standard procedure. Examination of these uneluted materials might be informative about how IFN-a2b isoforms behave in the column during elution. To this end, uneluted material was stripped from the column by using a low pH buffer (40 mM sodium acetate, 20 mM NaCI, pH 4), and fractions were assayed by RP-HPLC for isoform 1 and isoform 4 (FIG. 5A). These two isoforms were the predominant materials stripped from the column as shown by SDS-PAGE (FIG. 5B). The amounts of isoforms 1 and 4 contained in the eluted and stripped fractions were determined and the results shown that the vast majority of total isoform 1 was eluted from the column, while almost 2X more isoform 4 remained in the column than were eluted (data not shown). Thus, isoform 4 had greater affinity than isoform 1 for the DEAE column when the standard procedure was used.

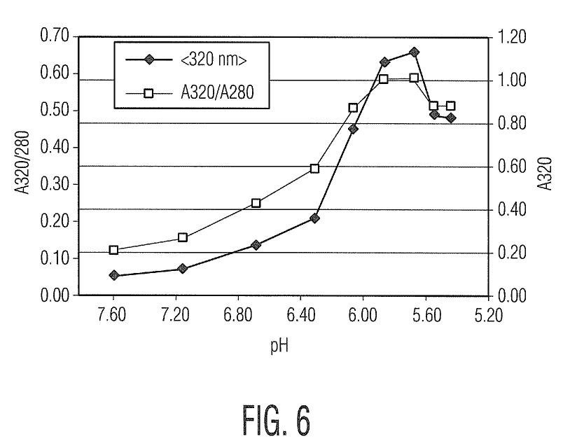

To understand why the uneluted materials bind to the column so tightly, we examined some properties of these materials. FIG. 6 shows determination of absorbance at OD320 and OD3201280 after the stripped material was exposed to various pHs. OD320 is a useful parameter indicative of aggregation or precipitation. OD320 or OD320/0D280 markedly increased as pH decreased to around 6, indicating that aggregation had occurred at pH of approximately 6. Aggregation at low pH may partially account for the strong binding of these materials to the column during elution.

Based on the above data, we concluded that IFN-a2b isoform separation on the DEAE Sepharose column occurs by two distinct mechanisms---chromatofocusing due to formation of a pH gradient, and selective binding of isoform 4 to the column under standard elution conditions.

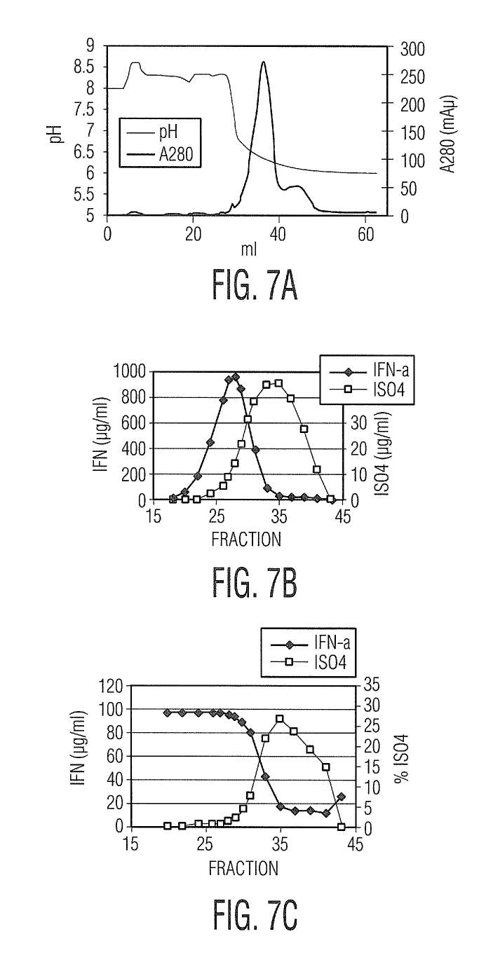

To reduce cycle times for the loading, wash and elution steps, we examined the possibility that the column size could be scaled down to a smaller DEAE Sepharose FF column (0.5 cm x 20 cm, 3.9 ml). The small column was able to generate a similar pH gradient as the 23 ml column and the UV profile for IFN-a2b, and separation of isoforms 1 and 4 using different elution buffers were also similar to the results obtained for the larger column (FIG. 7). Thus, most of the experiments described below were carried out by using a 0.5 cm x 20 cm, 3.9 ml column.

E. Effects of buffer concentration: relationship between pH gradient slope and resolution

In chromatofocusing procedures, column performance can frequently be optimized by manipulating several variables, including the range and slope of the pH gradient. To explore whether a narrower pH range with a shallower gradient would be useful in improving resolution of isoforms 1 and 4, we examined the effects of the phosphate concentrations in the elution buffer on the pH gradient slope and isoform resolution.

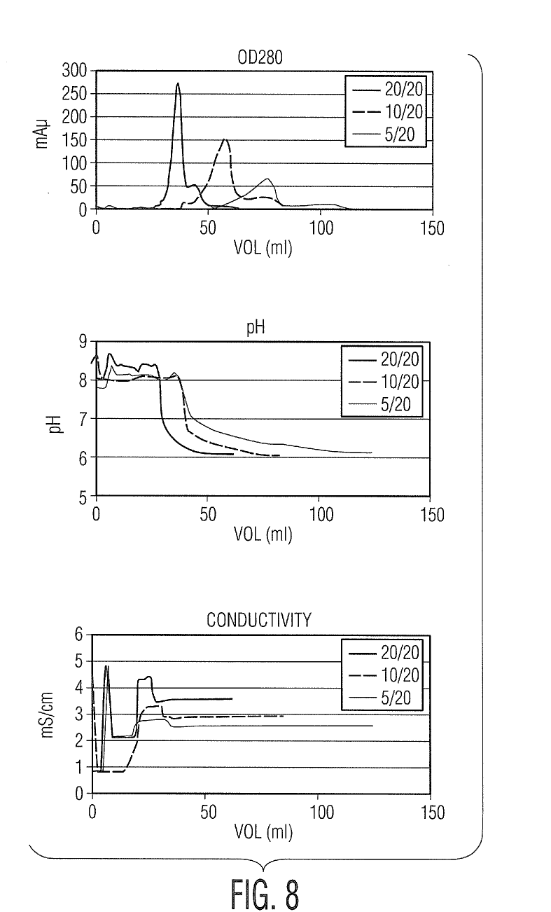

Elution was performed using 20, 10, or 5 mM sodium phosphate in the presence of 20 mM NaCI, pH 6.0. FIG. 8 shows the overlay of chromatography profiles obtained with each of these buffers. Clearly, as sodium phosphate concentration decreased, elution of IFN peaks from the column was delayed and the peak broadened (top panel). As expected, at low buffer concentrations, 10 or 5 mM sodium phosphate, formation of the pH gradient was also delayed and the slope was shallower than observed with the standard 20/20 elution buffer (FIG. 8, middle panel).

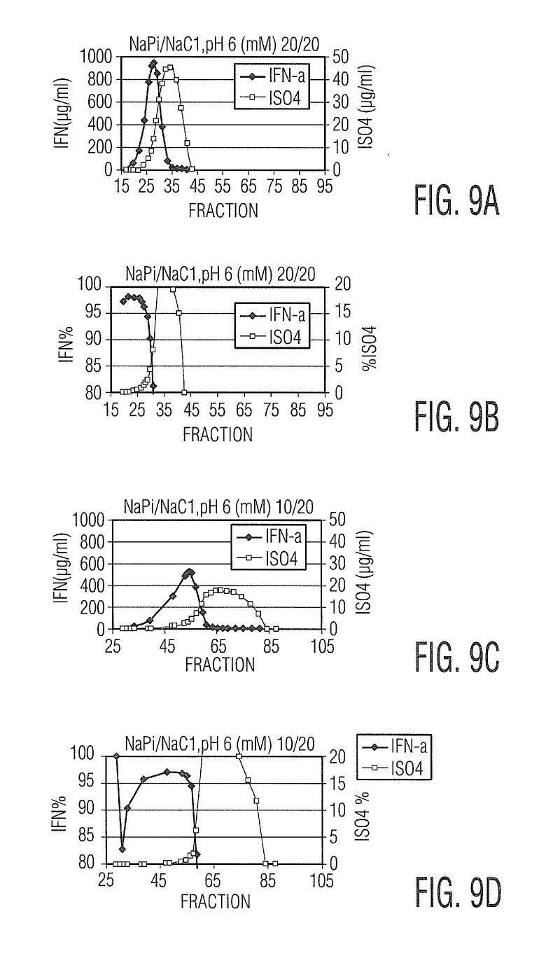

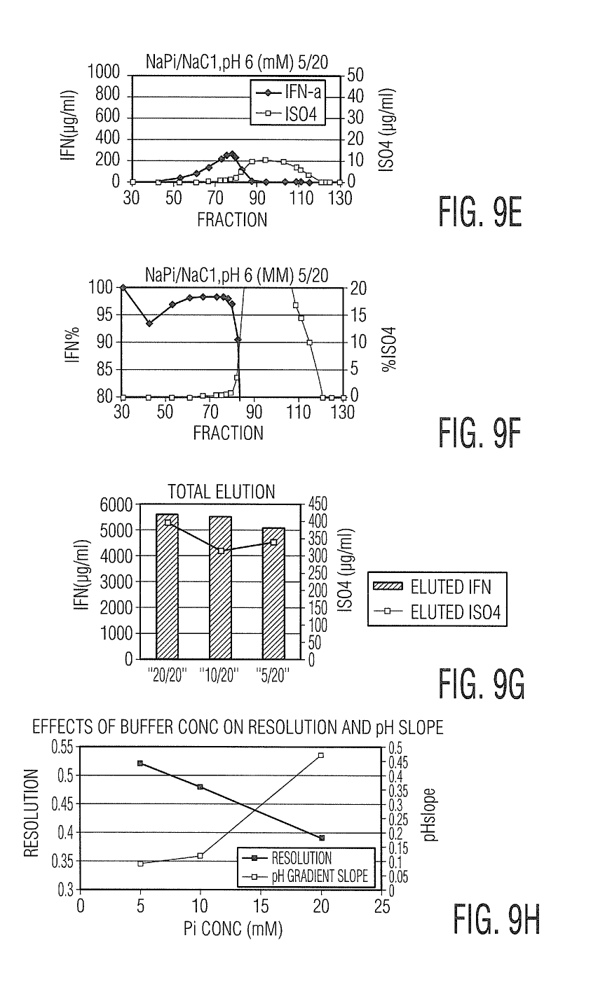

RP-HPLC assays demonstrated the broadening of the peaks for both isoforms 1 and 4, but improving separation of these isoforms, as the elution buffer phosphate concentration was reduced from 20 to 5 mM (FIG. 9, top 3 left panels). The total amounts of isoform 1 and isoform 4 that eluted were similar for each of the 3 phosphate concentrations (FIG. 9, bottom left panel).

The relationship among phosphate concentration, pH gradient and isoform resolution is shown in the bottom right panel of FIG. 9. The significant improvement in isoform resolution with reduced phosphate concentrations is consistent with conventional chromatofocusing, which shows the best resolution at lowest mobile buffer concentrations. The reason for this is that low buffer concentration helps to generate a shallow pH gradient, which in turn increases the resolution.

F. Impact of salt and phosphate concentration on IFN-a2b isoform 4 binding

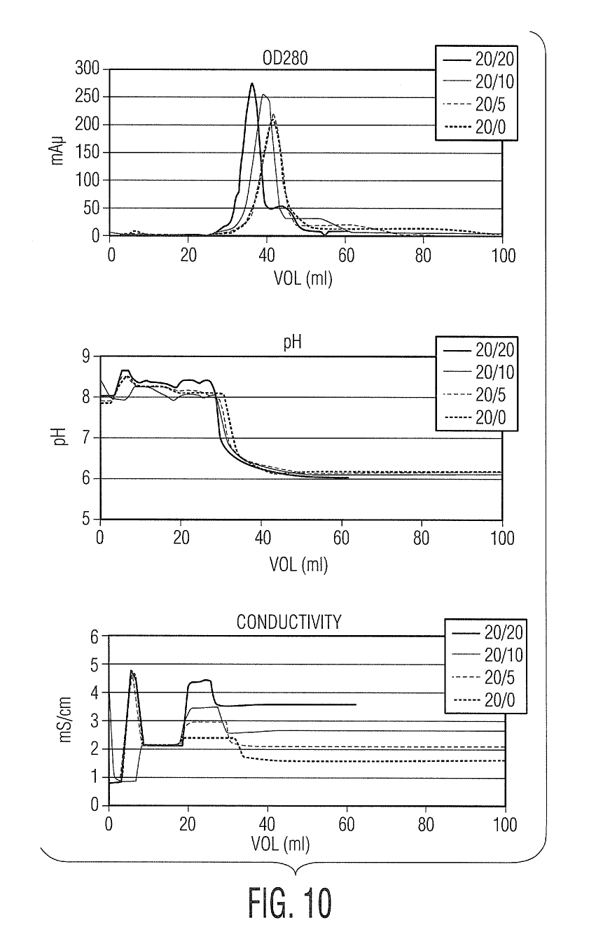

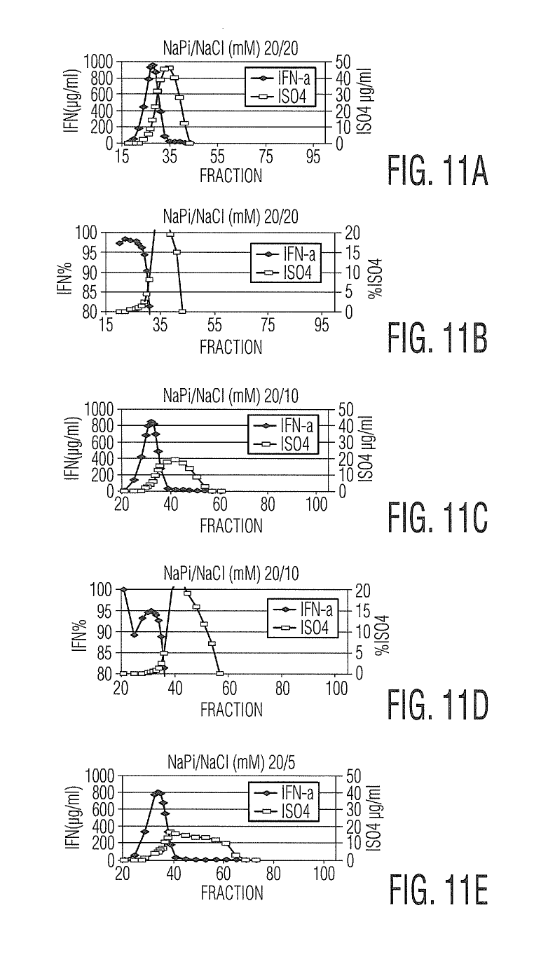

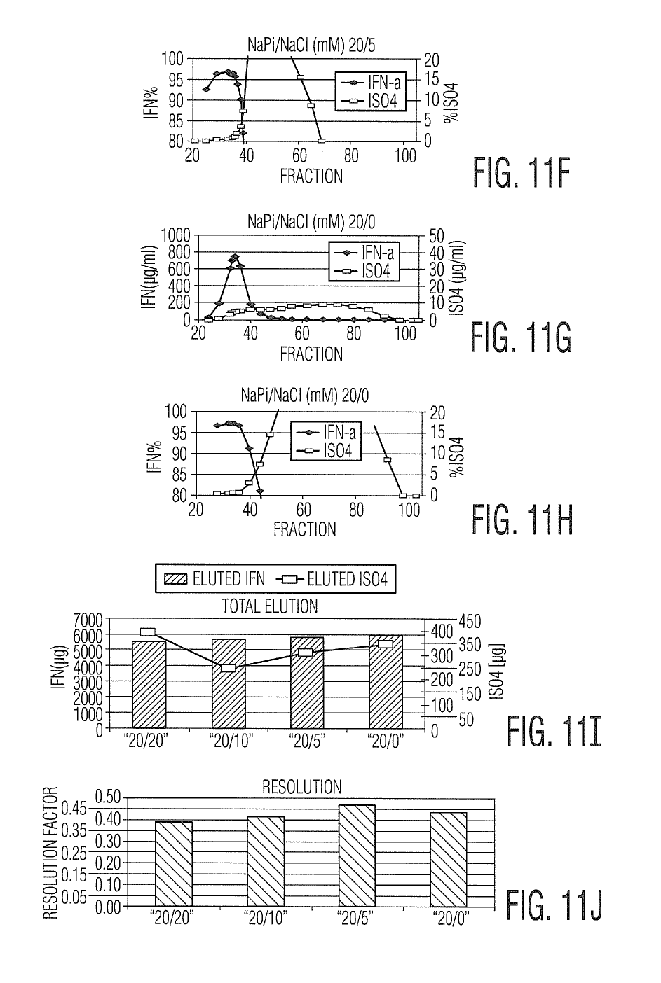

We examined the effect of ionic strength on IFN elution by performing elution using a pH 6.0 elution buffer containing 20 mM sodium phosphate and NaCi at 20 mM, 10 mM, 5 mM or 0 mM. The results are shown in FIGS. 10 and 11.

Elution of isoform 1 was little affected by NaCl concentration in the 20 mM phosphate buffer in terms of peak position and sharpness (FIG. 10, top panel; FIG. 11, left top 4 panels. However, the isoform 4 peak progressively broadened as NaCI concentration decreased (FIG. 11, left top 4 panels), indicating that the rate of isoform 4 elution from the column was reduced in the absence of salts. The pH appeared relatively unchanged by salt concentrations (FIG. 10 middle panel). In the pooled fractions, the total amount of isoforms 1 and 4 eluted from the column and their resolution were similar under different salt conditions.

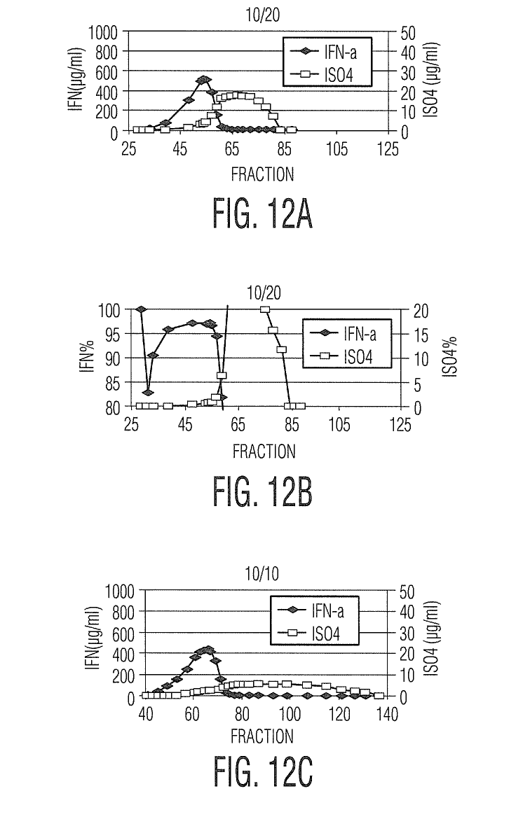

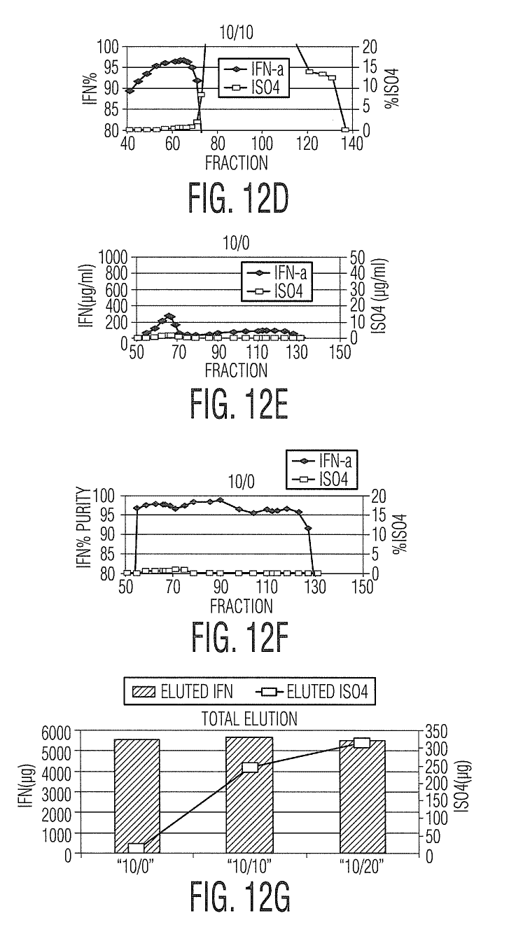

Since we had determined that resolution of isoforms 1 and 4 could be improved at low buffer concentrations during elution, we assessed the effect of reducing salt concentration (either 20 mM, 10 mM or 0 mM NaCI) in a 10 mM sodium phosphate, pH 6.0 elution buffer. The results are shown in FIG. 12.

Similar to the results observed using the 20 mM sodium phosphate elution buffer, the isoform 4 peak eluted with the 10 mM sodium phosphate buffer broadened as the NaCI concentration decreased from 20 to 10 mM while the isoform 1 peak was little affected (FIG. 12, top 2 left and right panels). However, as salt concentration was further reduced to 0 mM, isoform 1 eluted as two distinct peaks (FIG. 12, 3^{rd} left and right panels), but only a small amount (1.6%) of the total isoform 4 co-eluted with the first isoform 1 peak (FIG. 12, 3rd right and left panels). In contrast, 36% of the total isoform 4 co-eluted with the isoform 1 peak using the standard 20 mM sodium phosphate/20 mM NaCl/pH 6.0 elution buffer.

Quantitative analysis indicated that the total amount of isoform 1 eluted from the column was unchanged by the salt concentration at 10 mM sodium phosphate, while isoform 4 elution was greatly suppressed as the salt concentration was reduced (FIG. 12, bottom left panel). These results suggest that the DEAE Sepharose FF column has more affinity to isoform 4 than isoform 1 when elution is performed using a low buffer concentration with no salt. Purity analysis by RP-HPLC assay indicated that almost all IFN fractions had a low percentage of isoform 4 (<1%) (FIG. 12, bottom right panel).

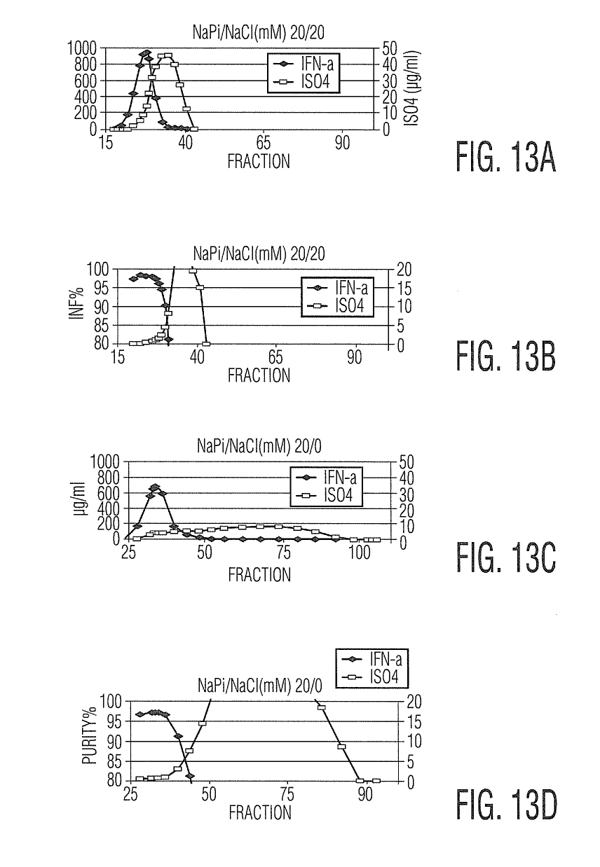

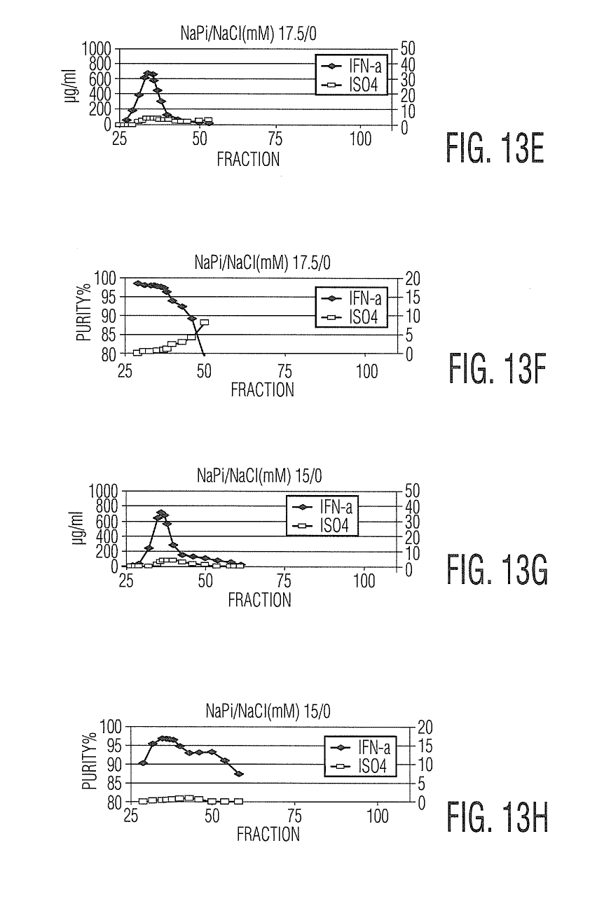

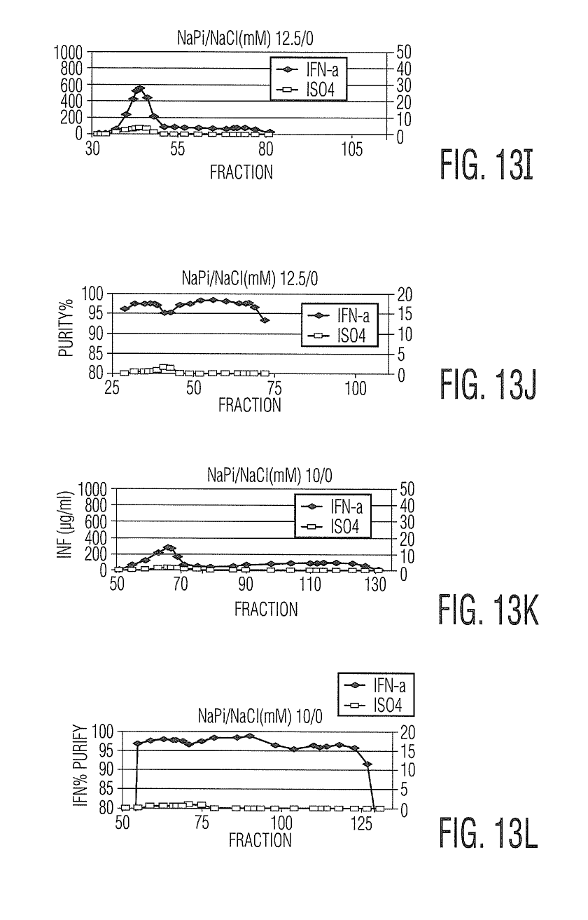

To further examine binding of IFN-α2b isoforms to the column at low buffer concentrations, we performed elution in a pH 6.0 buffer with sodium phosphate concentration at 20, 17.5, 15, 12.5 or 10 mM and no salt. The results are shown in FIG. 13.

Compared to elution with the standard 20/20 buffer, the isoform 4 peak at 20 mM sodium phosphate/0 mM NaCI was broadened but its total elution was unaffected. As sodium phosphate reduced from 20 to 17.5, 15, 12.5, and 10 mM, elution of isoform 4 was increasingly delayed or blocked with corresponding broadening of the isoform 1 peak (FIG. 13, left panels). RP-HPLC assays revealed that almost all of the isoform 1 fractions eluted using low buffer concentrations (5_{—} 15 mM sodium phosphate) contained a very low percentage of isoform 4. A transition point for isoform 4 binding to the column occurred between 15 and 20 mM of sodium phosphate, likely due to increased isoform 4 precipitation at this phosphate concentration.

### G. Effect of pH

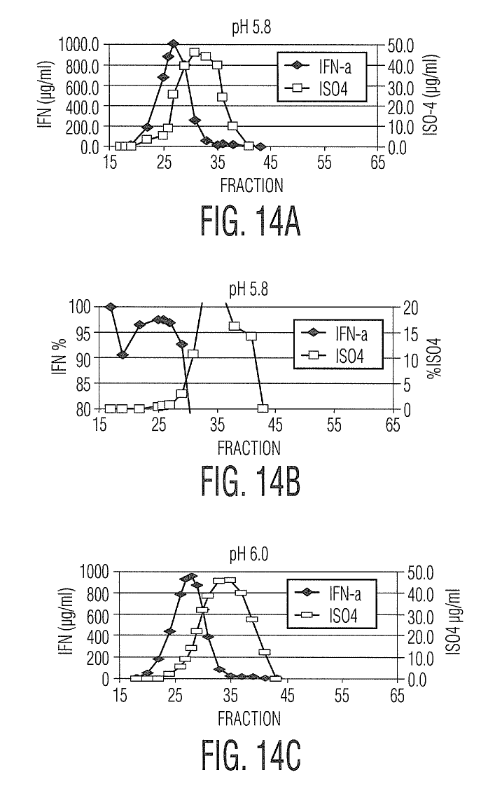

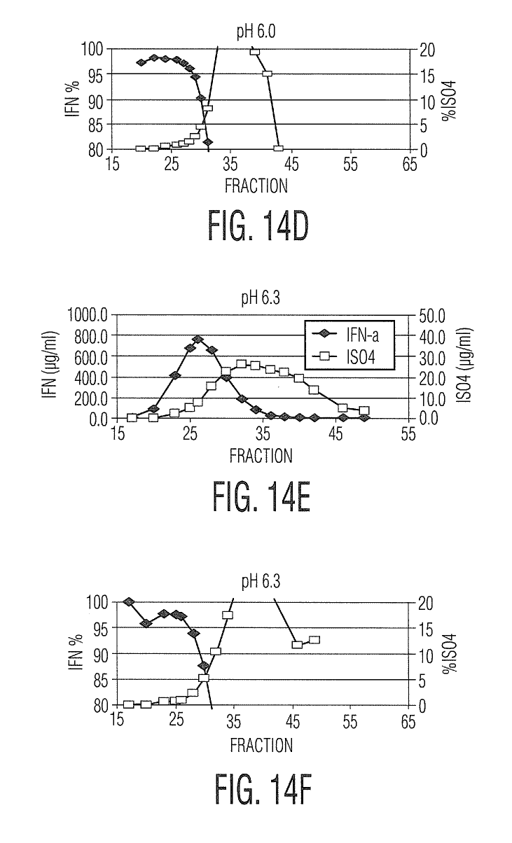

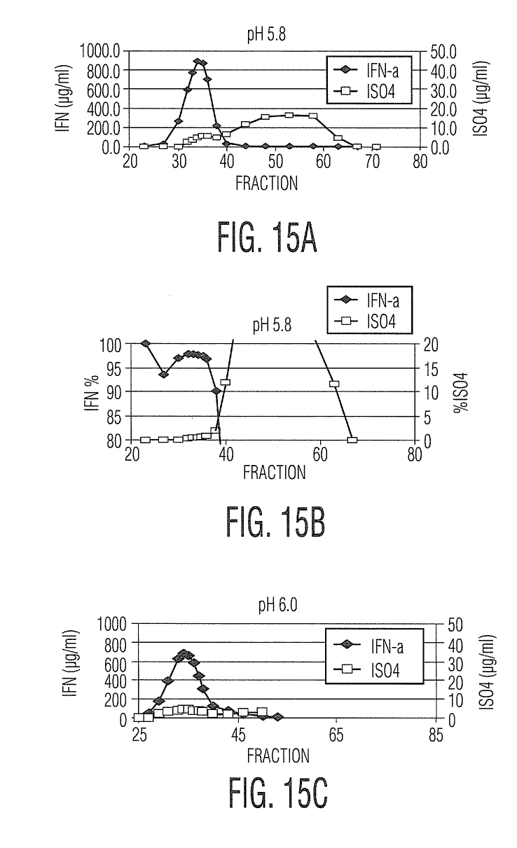

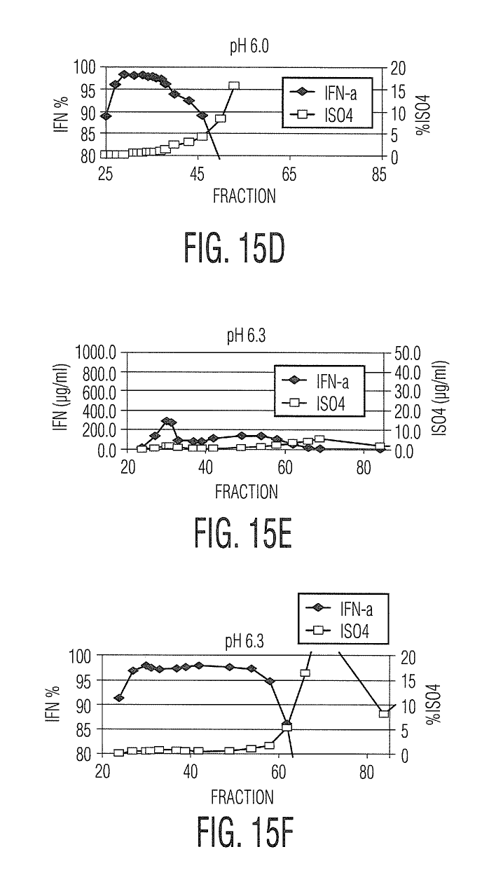

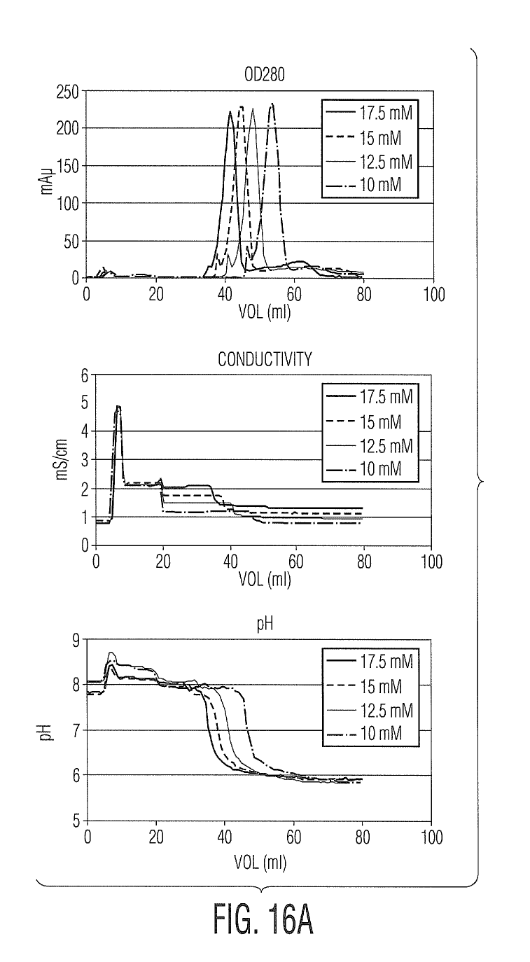

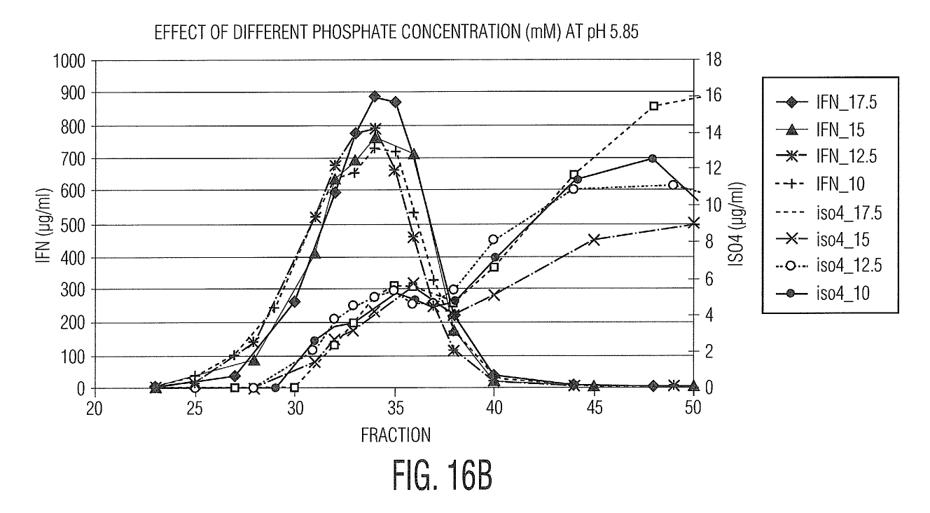

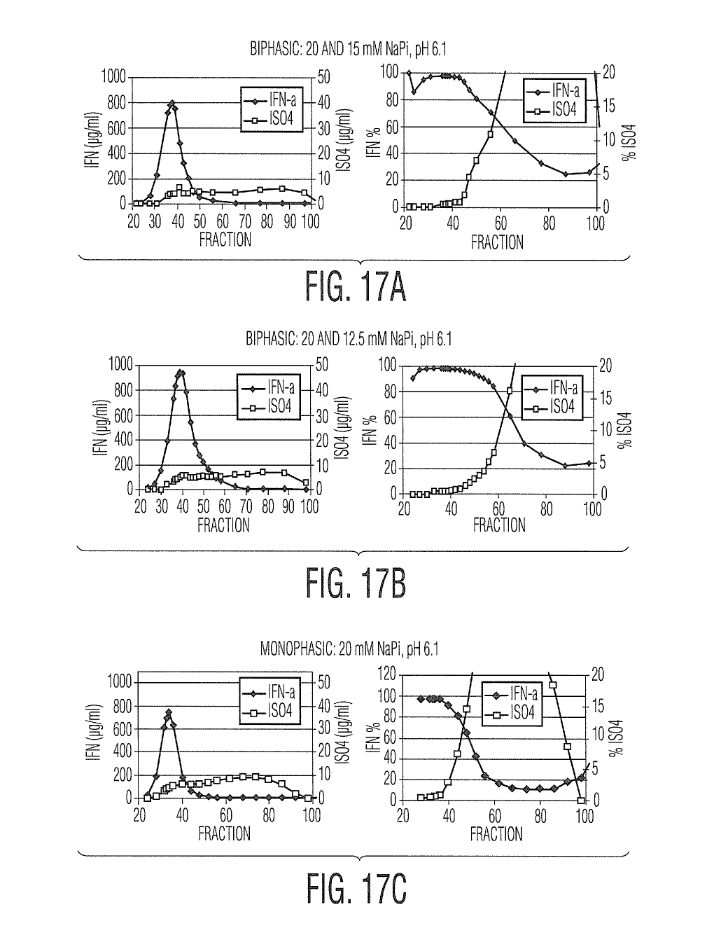

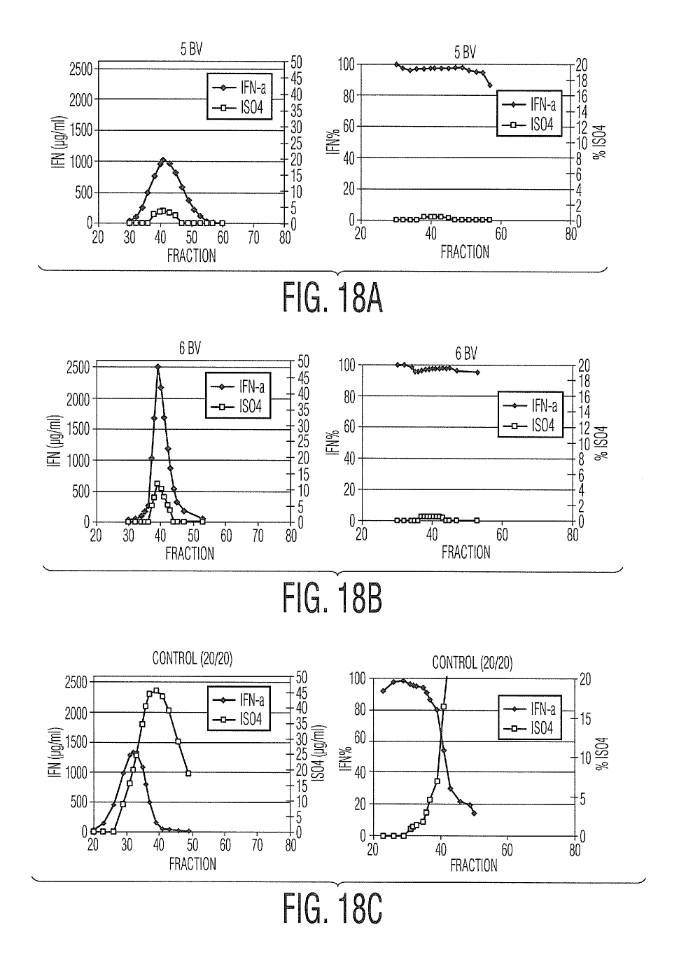

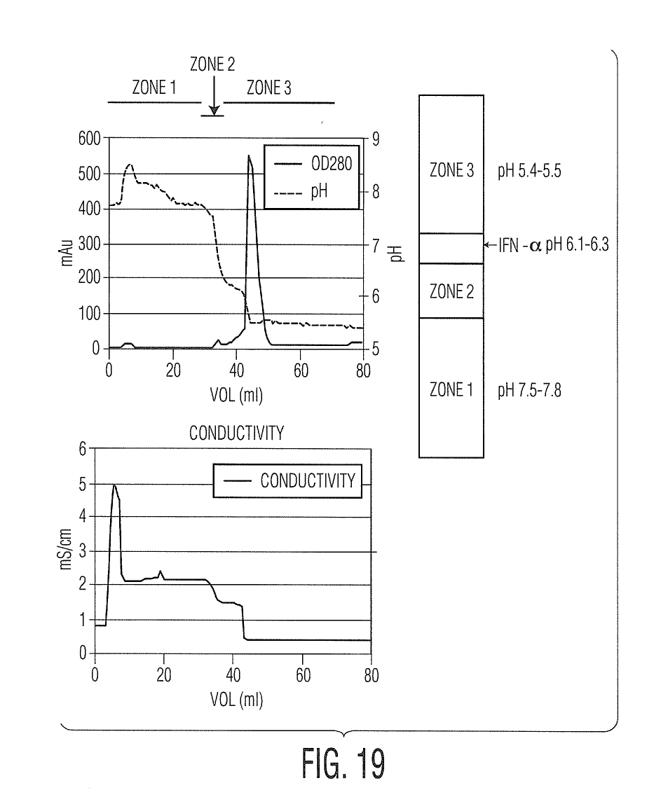

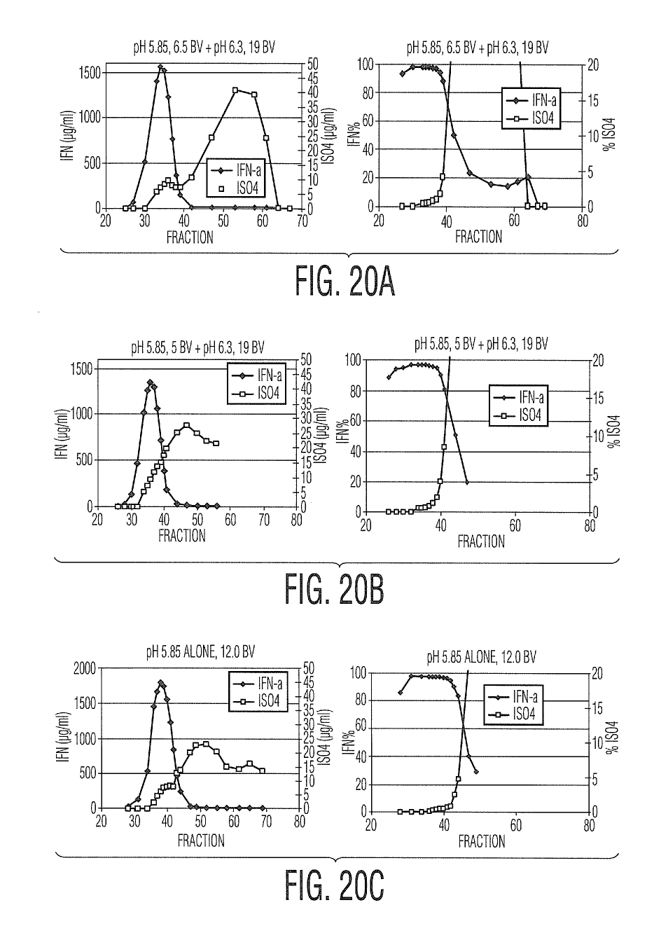

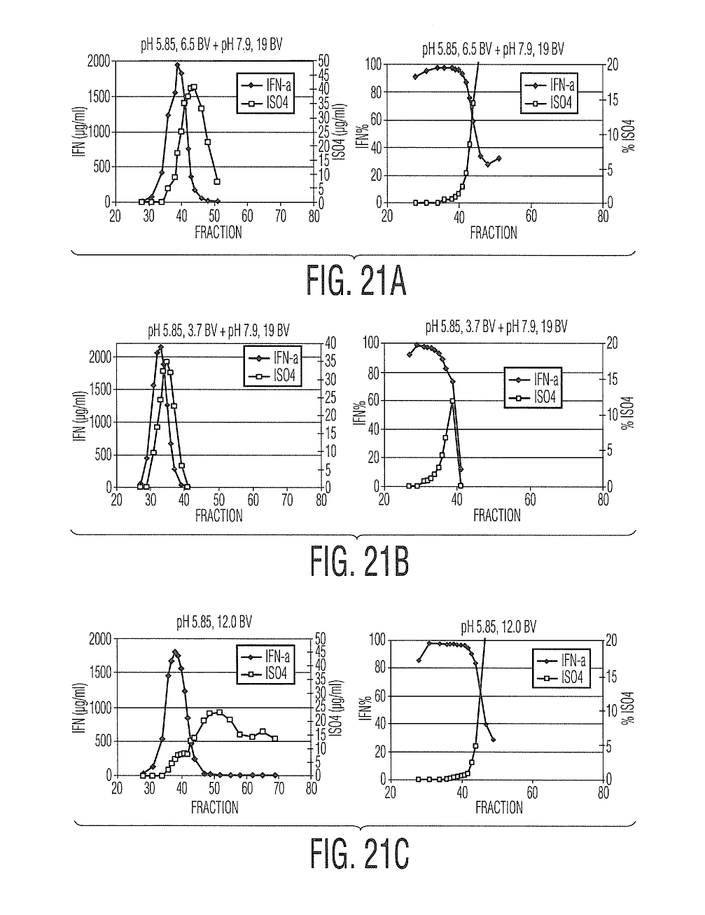

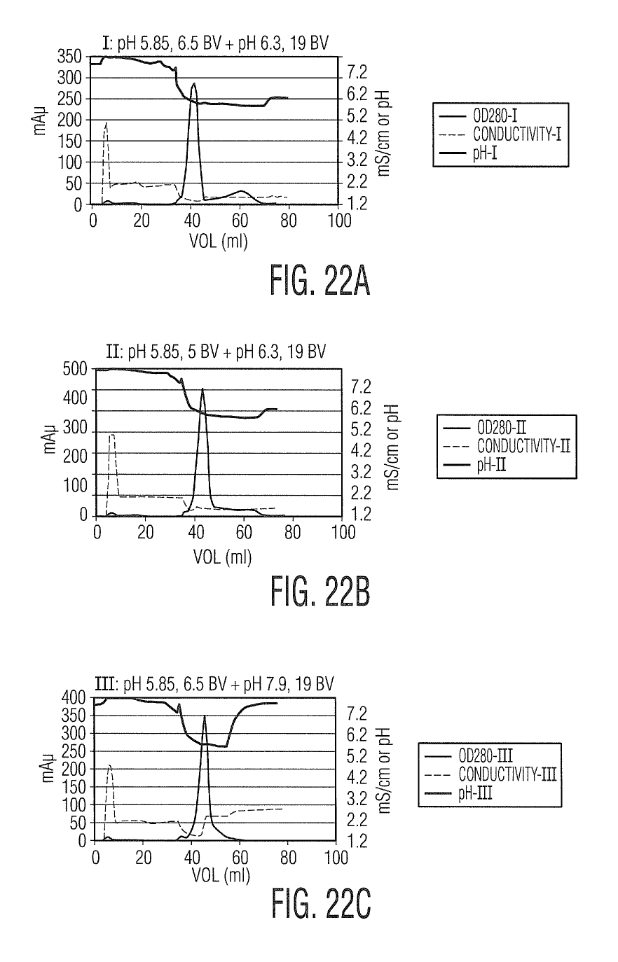

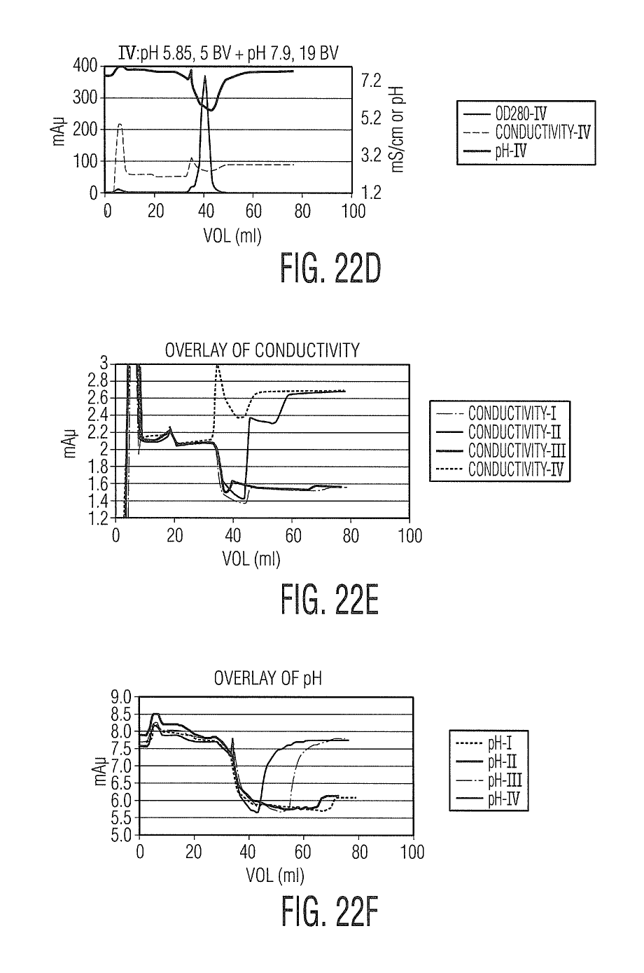

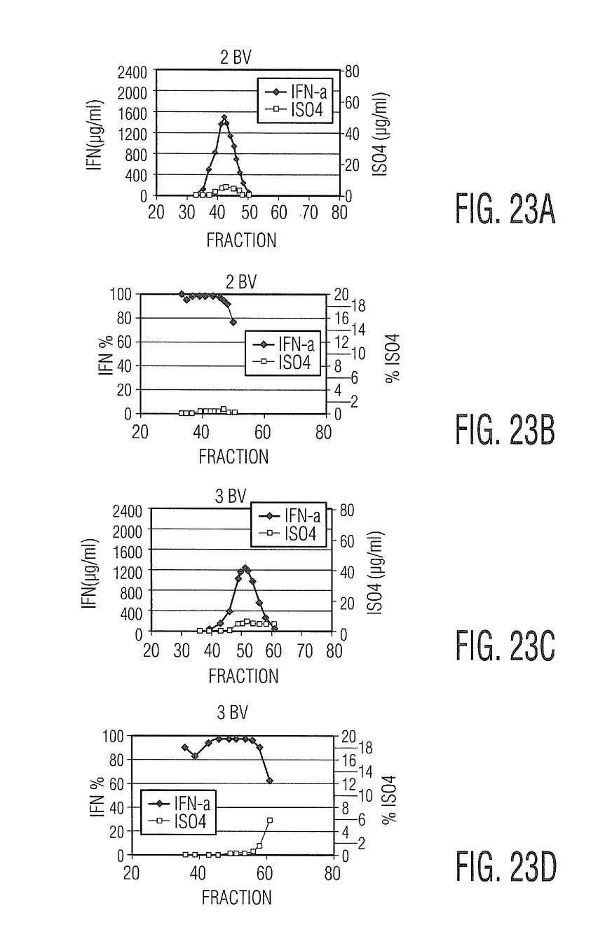

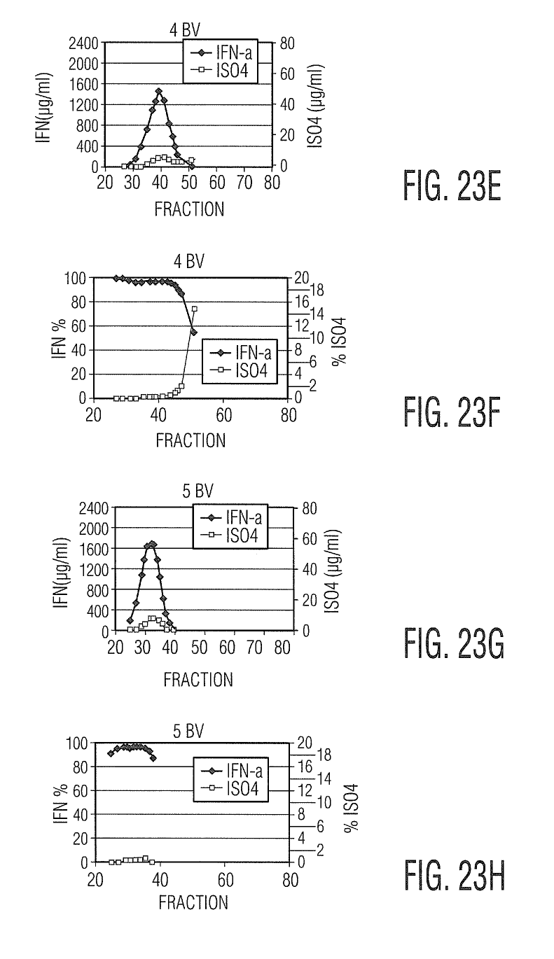

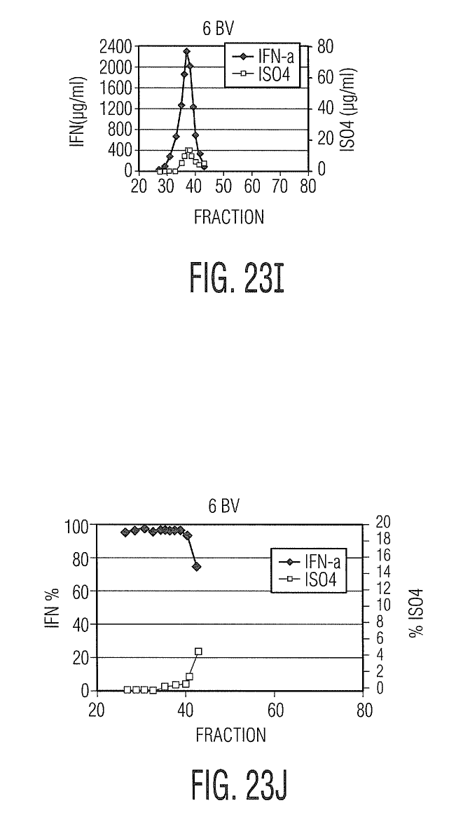

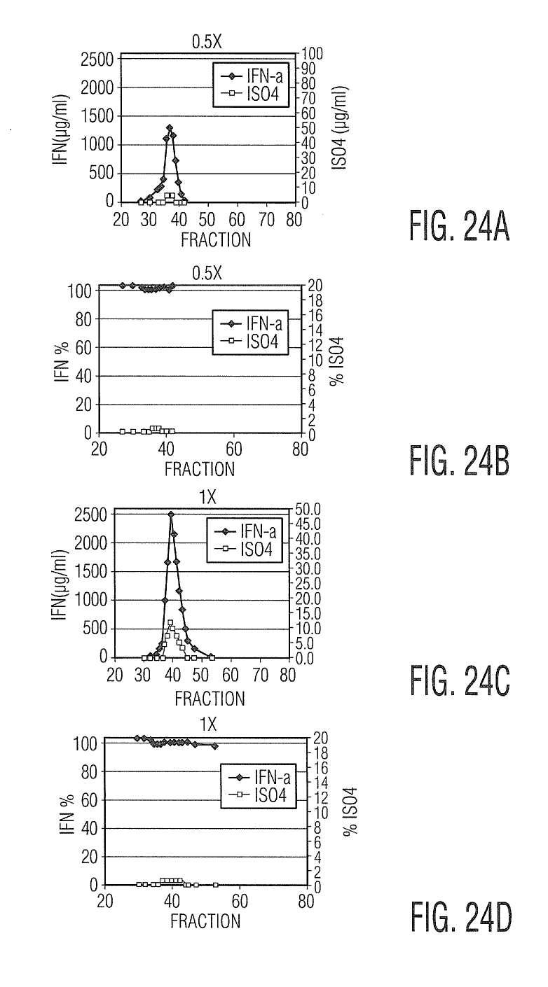

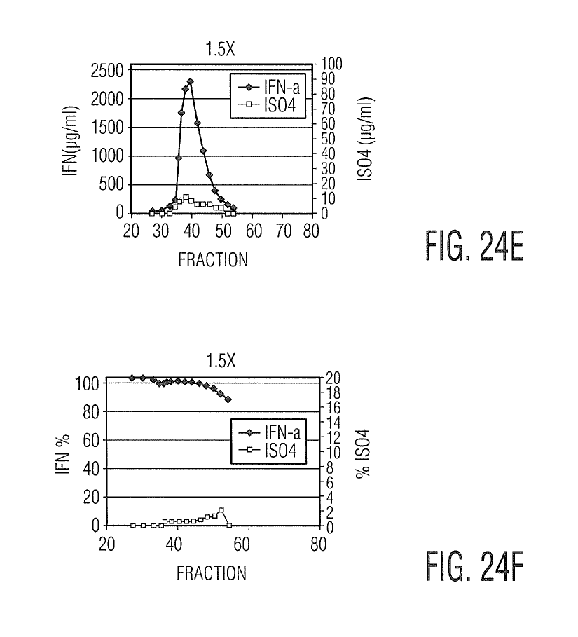

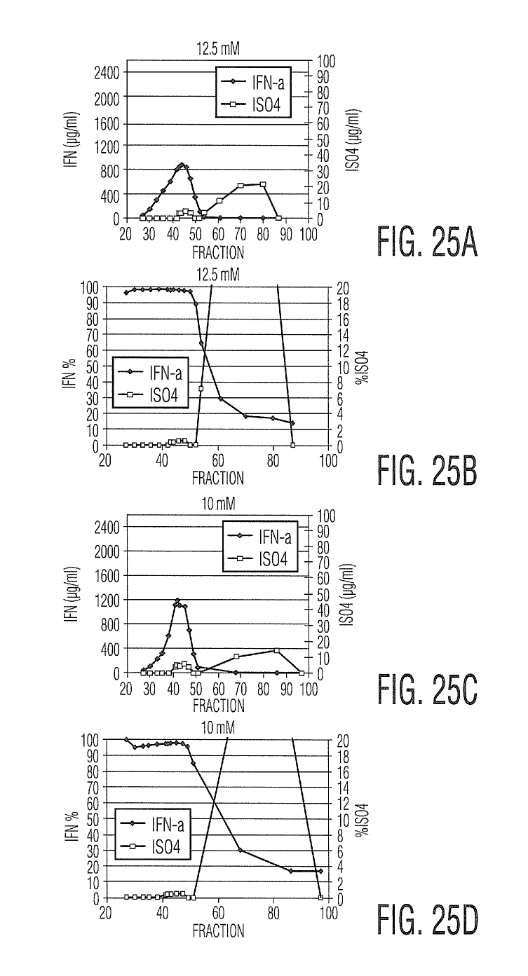

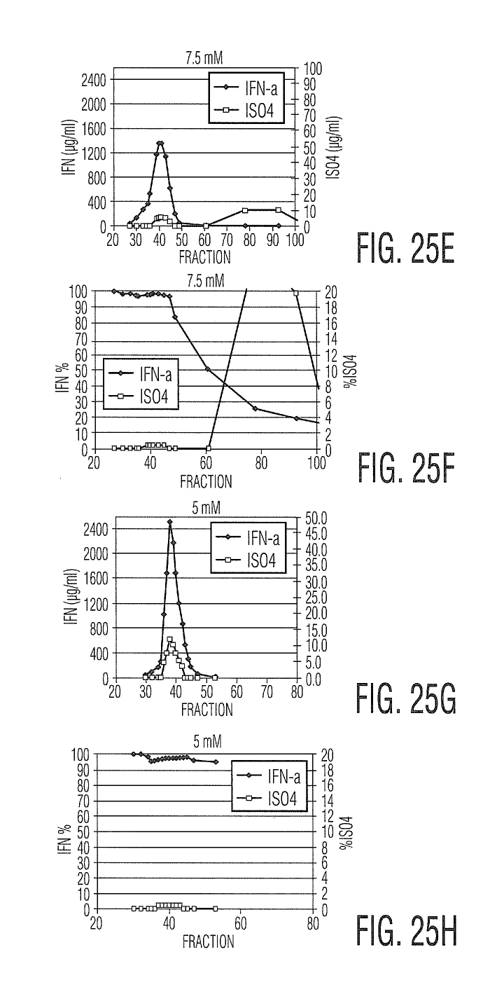

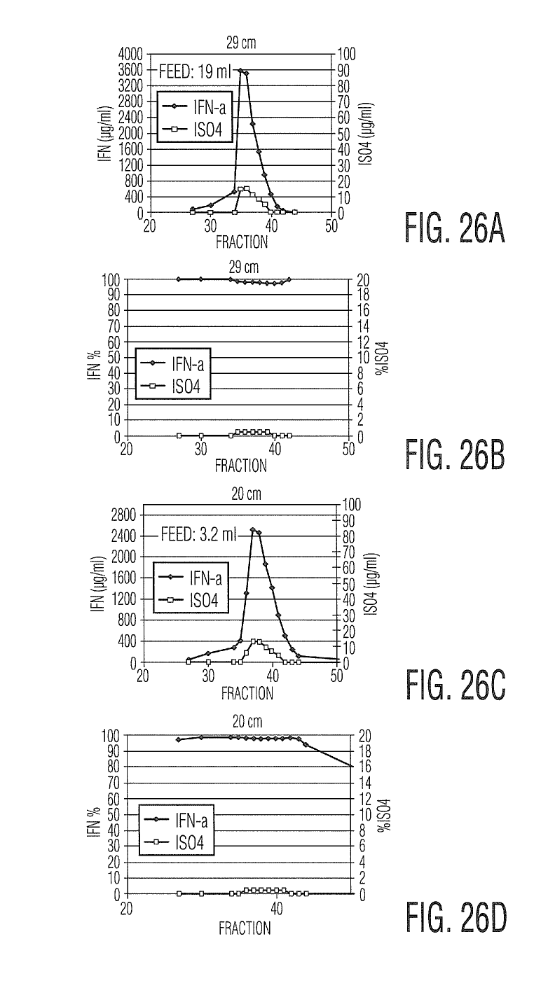

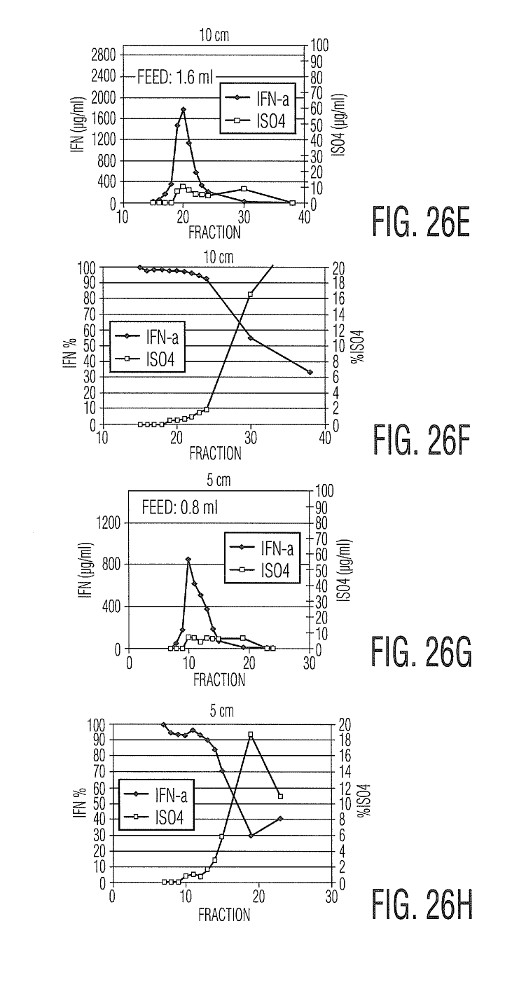

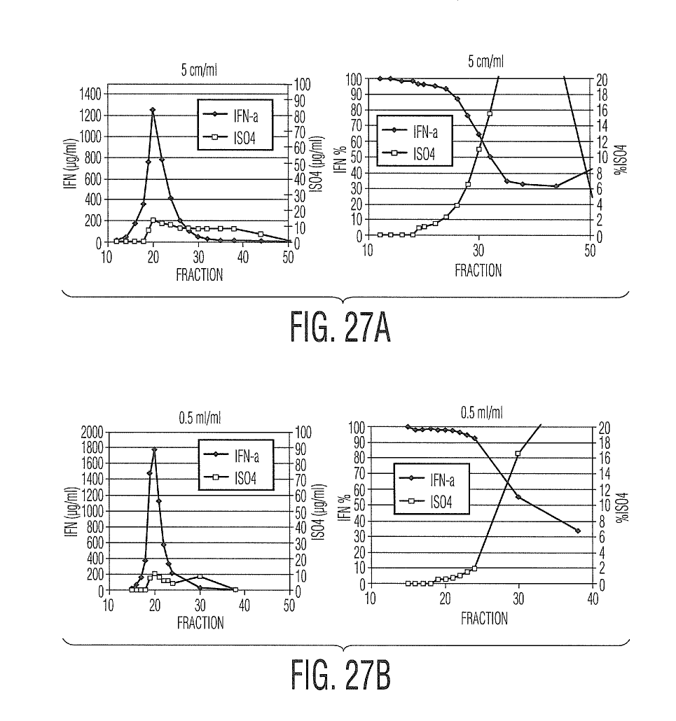

Because pH influences the net charge and distribution of surface charges on protein molecules, changes the anionic makeup of the mobile buffer and may modulate the charge status of anion exchange resins, we assessed the effect of varying pH from 5.8 to 6.3 on elution of IFN-a2b isoforms 1 and 4 with 20 mM sodium phosphate/20mM NaCI (20/20) or 17.5 mM sodium phosphate/0 mM NaCI (17.5/0) elution buffers. The results are shown in FIGS. 14 and 15.

Elution using the 20/20 buffer produced very similar profiles for both isoforms at pH 5.8, 6.0 and 6.3; however, a broadening of each isoform peak was observed at pH 6.3 (FIG. 14). The total amount of each isoform that eluted from the column was also very similar at each pH (data not shown). These results indicated that IFN-α2b elution using standard buffer and salt concentrations was relatively insensitive to buffer pH.

In contrast, when elution was performed with 17.5 mM sodium phosphate/0 mM NaCI, varying the pH between 5.8 and 6.3 had significant effects. Little isoform 4 eluted at pH 6.0 (FIG. 15, middle panel). However, at pH 5.8, the isoform 1 peak became sharper and significantly more isoform 4 eluted from the column (FIG. 15, top panel). At pH 6.3, isoform 1 eluted as two peaks, and basically no isoform 4 eluted (FIG. 15, bottom panel). Therefore, IFN-a2b elution was very sensitive to relatively small pH changes at 17.5 mM sodium phosphate in the absence of NaCI.

H. Total and poolable recoveries

The total elution of isoform 1 under all conditions described above was very similar and close to 100%, while isoform 4 elution was dramatically reduced at sodium phosphate 17.5 mM in the absence of NaCl. However, the improved resolution of these isoforms achieved with elution buffers containing 17.5 mM sodium phosphate/0 mM NaCI as compared to elution with the standard 20/20 buffer has a significant efficiency cost: a significantly larger volume of fractions would need to be pooled to achieve high isoform 1 yields, e.g. ≧90%, with low isoform 4 contamination, e.g., <3%, due to the slowed or delayed elution of IFN peaks.

Thus, additional experiments were performed to attempt to identify elution conditions that would achieve high resolution of isoforms 1 and 4.

### III. Results and Discussion—Experimental Series II

In the second series of experiments, several different substantially purified IFN-α2b preparations were used, and are listed below.

| IFN preparation | Isoform 1 (mg/ml) | Isoform 4 (mg/ml) |
|---|---|---|
| 1 | 1.77 | 0.25 |
| 2 | 3.49 | 0.50 |
| 3 | 3.42 | 0.63 |
| 4 | 2.55 | 0.48 |
| 5 | 3.56 | 0.52 |

A. Effect of buffer and IFN concentration on elution volume at pH 6.0

To assess the effect on elution volume of buffer and IFN concentration at pH 6.0, we compared elution volumes obtained for two different IFN-a2b concentrations (1.77 mg/ml and 3.49 mg/ml) using the standard 20 mM sodium phosphate/20 mM NaCl elution buffer to elution volumes obtained for these IFN concentrations using lower buffer concentrations in the absence of salt. The results are shown in table IIIA below.

TABLE IIIA
Elution volumes for isoform 1 peak under different conditions

| Elution conditions | IFN Preparation 1 | IFN Preparation 2 |
|---|---|---|
| (NaPi/NaCl, mM) | (1.77 mg/ml Iso 1) | (3.49 mg/ml Iso 1) |
| 20/20 | 20 ml (5.1 BV) | 23 ml (5.9) |
| 15/0 | 27 ml (6.9 BV) | 48 ml (12 BV) |
| 12.5/0 | 39 ml (10 BV) | 61 ml (16 BV) |
| 10/0 | 64 ml (16 BV) | Not determined |

For IFN preparation 1, the elution volume for the isoform 1 peak increased by 35%, 95%, and 220%, with elution at 15, 12.5, and 10 mM phosphate, respectively, compared to the elution volume using the standard 20/20 buffer (Table IIIA, 2^{nd} column). A similar pattern of increasing elution volume with decreasing sodium phosphate concentration was observed for the IFN preparation that contained almost twice as much IFN (Table IIIA, 3^{rd} column). However, the magnitude of this effect of sodium phosphate concentration on elution volume was increased at the higher IFN concentration. The IFN concentration did not appreciably affect the resolution of isoforms 1 and 4 in buffer lacking NaCI, as isoform 1 from either batch eluted as two broad peaks with little co-elution of isoform 4, the majority of which remained on the column (data not shown).

B. IFN elution volume was independent of buffer concentration at pH 5.85

As discussed in Section II.G above, the pH displayed noticeable effects on IFN elution at 17.5 mM sodium phosphate concentrations in the absence of NaCI in the elution buffer, with a pH 5.85 elution buffer producing isoform 1 as a sharp peak and increasing isoform 4 elution while a pH 6.3 elution buffer suppressed isoform 4 elution but generated a broad isoform 1 peak. To examine the relationship of buffer concentration and isoform elution at pH, we performed the standard DEAE chromatography procedure on IFN preparation 1 using elution buffers containing no salt and sodium phosphate at 17.5 mM, 15 mM, 12.5 mM or 10 mM at pH 5.85. The results are shown in FIGS. 16A and 16B.

The elution of IFN was progressively delayed as the phosphate buffer concentration decreased from 17.5 to 10 mM (FIG. 16A, top panel). As expected, the conductivity reduction and pH gradient formation were also delayed at lower buffer concentrations (FIG. 16A, middle and bottom panels). However, when the IFN elution profiles were overlayed (the IFN peaks for each buffer concentration were normalized to the same fraction for comparison) we observed that the shape of the isoform 1 peak was sharp and remarkably similar among the tested phosphate concentrations, while isoform 4 was increasingly eluted in the later fractions of the IFN peak (FIG. 16B). The observation that lowering the buffer concentration at low pH (5.85) did not significantly broaden isoform 1 peak as it would at higher pH (6.0-6.2), suggested that a lower pH and lower concentration of buffer might be useful for elution of isoform 1 without dramatically increasing the elution volume.

IV, Biphasic elution—Rationale, Results and Discussion

None of the above test procedures provided satisfactory separation of isoform 1 from isoform 4 coupled with elution of isoform 1 in an acceptably small volume. A larger isoform 1 pool volume was always obtained with elution conditions, such as lower phosphate buffer concentrations, that either facilitated isoform 4 binding or improved the separation of isoforms 1 and 4. On the other hand, while we could obtain a sharper isoform 1 peak and thus smaller pooled elution volumes with higher phosphate buffer concentrations or lower pH, more isoform 4 co-eluted with isoform 1 thereby reducing the yield of isoform 1 that met the desired purity criteria. In the first series of experiments, we observed that isoform 4 did not start to elute in most cases until the first quarter or half peak of isoform 1 had eluted.

Based on these observations, we hypothesized that changing the elution buffer to a lower concentration buffer or high pH buffer following elution of the first half isoform 1 peak, i.e. via a two-step or “biphasic” elution procedure, suppression of isoform 4 elution would occur in the second half of the isoform 1 peak, thus allowing isoform 1 to be eluted in a smaller elution volume. To test this hypothesis, the standard DEAE chromatography procedure described above was performed on the 3.9 ml column except that the single elution step was replaced with a two-step, i.e., biophasic, elution process. In step 1, a strong elution solution was applied to the column following the wash step and in step 2 a weaker elution solution with a different composition was applied. The flow rates were the same as used for the standard single elution step. The results obtained with various compositions for the strong and weak elution solutions are described below.

A. Biphasic elution with different phosphate concentrations at the same pH.

1. First phase elution with 20 mM phosphate, pH 6.1; second phase elution with 15 or 12.5 mM phosphate, pH 6.1.

A biphasic elution of IFN-a2b (IFN preparation 2) was performed with 6.7 bed volumes of the strong elution solution (20 mM sodium phosphate, pH 6.1), followed by 19 bed volumes of the weak elution solution (15 mM or 12 mM sodium phosphate, pH 6.1). The results are shown in FIG. 17.

Isoform 1 was efficiently eluted from the column, with significantly improved separation from isoform 4 as compared to elution performed using the standard 20/20 conditions (FIG. 17, top two panels). However, the RP-HPLC analysis profile looked very similar to that obtained using a single high phosphate (20 mM sodium phosphate) elution buffer at pH 6.1 (FIG. 17, bottom panel). Some isoform 4 eluted within later fractions of the second half of the isoform 1 peak, which could make those fractions not poolable depending on the desired purity criteria. No significant difference was observed in the isoform elution profiles generated by the different elution solutions, except that the isoform 1 peak width eluted using 12.5 mM sodium phosphate (FIG. 17, middle panel) was larger than elution with 15 mM sodium phosphate. Hence, biphasic elution using two different phosphate concentrations (15 0112.5 mM) at pH 6.1 did not appear optimal. 2. First phase elution with 17.5 mM phosphate, pH 5.85; second phase elution with 5 mM phosphate, pH 5.85.

A very different profile was obtained when biphasic elution of IFN-a2b (IFN preparation 5, loading volume of 3.2 ml) was performed using two different phosphate concentrations at pH 5.85. The first elution phase was carried out with 5 or 6 B.V of 17.5 mM sodium phosphate, pH 5.85 followed by elution with 19 bed volumes of 5 mM sodium phosphate, pH 5.85. The results are shown in FIG. 18.

In both experiments, essentially every fraction eluted across the isoform 1 peak with was greater than 97% purity, with less than 0.5% isoform 4 (FIG. 18). However, isoform 1 eluted as a much sharper peak when the first phase elution was performed with six instead of five B.V. of strong elution solution. The isoform 1 peak fraction was 2.5 mg/ml IFN-α2b, nearly twice the IFN concentration of the peak fraction (1.3 mg/ml) obtained in the standard procedure. The table below shows a detailed fractional analysis comparing the yield and purity achieved with the standard single phase and the biphasic experiment using 6 B.V. of strong elution solution.

TABLE IV.B
Comparison of Standard and Biphasic Elution Procedures

| Standard Procedure | | | | | Biphasic Procedure | | | | |
|---|---|---|---|---|---|---|---|---|---|
| | Conc (ug/ml) | | Purity (%) | | | Conc (ug/ml) | | Purity (%) | |
| Fraction | ISO1 | ISO4 | ISO1 | ISO4 | Fraction | ISO1 | ISO4 | ISO1 | ISO4 |
| 20 | 37.5 | 0.0 | 91.9 | 0.0 | 28.0 | 34.4 | 0.0 | 100.0 | 0.0 |
| 23 | 139.6 | 0.0 | 97.8 | 0.0 | 30.0 | 56.8 | 0.0 | 100.0 | 0.0 |
| 26 | 446.6 | 0.0 | 98.7 | 0.0 | 32.0 | 96.5 | 0.0 | 98.4 | 0.0 |
| 29 | 970.9 | 8.8 | 96.4 | 0.9 | 34.0 | 169.8 | 0.0 | 95.6 | 0.0 |
| 31 | 1281.9 | 15.5 | 95.6 | 1.2 | 35.0 | 264.2 | 0.0 | 95.9 | 0.0 |
| 32 | 1331.3 | 19.9 | 95.0 | 1.4 | 36.0 | 1030.1 | 4.9 | 96.3 | 0.5 |
| 33 | 1329.8 | 24.4 | 94.4 | 1.7 | 37.0 | 1684.4 | 7.6 | 97.0 | 0.4 |
| 35 | 1075.3 | 34.6 | 91.3 | 2.9 | 38.0 | 2512.9 | 12.0 | 97.3 | 0.5 |
| 36 | 782.2 | 40.4 | 86.8 | 4.5 | 39.0 | 2168.2 | 10.2 | 97.5 | 0.5 |
| 37 | 504.2 | 44.2 | 79.9 | 7.0 | 40.0 | 1680.7 | 7.7 | 97.5 | 0.4 |
| 39 | 151.5 | 45.4 | 54.6 | 16.4 | 41.0 | 1192.6 | 5.3 | 97.4 | 0.4 |
| 41 | 50.5 | 43.5 | 30.0 | 25.8 | 42.0 | 868.9 | 3.7 | 97.9 | 0.4 |
| 43 | 32.9 | 39.0 | 21.8 | 25.9 | 43.0 | 532.7 | 0.0 | 97.7 | 0.0 |
| 46 | 23.8 | 28.9 | 19.6 | 23.7 | 44.0 | 318.6 | 0.0 | 98.2 | 0.0 |
| 49 | 10.3 | 18.8 | 14.0 | 25.4 | 45.0 | 182.2 | 0.0 | 96.2 | 0.0 |
| | | | | | 47.0 | 61.3 | 0.0 | 95.4 | 0.0 |

The pH profile in the biphasic elution procedure using 6 B.V. of strong elution solution demonstrated several pH displacement zones formed along the length of the column during the elution (FIG. 19). These zones correlated to conductivity changes during elution (FIG. 19, lower panel). A sharp displacement front occurred between pH 5.5 and pH 6.1. Isoform 1 was rapidly eluted at pH 5.5 and moved down the column, and became focused at the pH displacement front. Therefore, formation of discrete pH fronts explained the fact that isoform 1 eluted as a sharp peak during biphasic elution.

It was noted that some precipitation, dissolvable by adjusting the pH or salt concentration, occurred particularly at room temperature in several fractions with high IFN concentrations, likely due to the low conductivity conditions in the elution buffer. The total recovery of isoform 1 in fractions pooled according to a desired purity criteria was as high as 99%. Similar performance was obtained with different IFN-α2b preparations and column lengths (data not shown).

C. Biphasic elution with different pH at the same phosphate concentration

We also examined the effect of holding the phosphate buffer constant while varying the pH in a biphasic elution procedure. A low pH buffer was used as the strong elution solution to sharpen elution of the isoform 1 peak. A high pH buffer was then employed to inhibit isoform 4 elution.

1. First phase elution with 17.5 mM phosphate, pH 5.85; second phase elution with 17.5 mM phosphate, pH 6.3.

The DEAE column was loaded with IFN preparation IFN preparation 4 and washed according to the standard procedure. Biphasic elution was performed using 6.5 B.V. or 5.0 B.V. of 17.5 mM sodium phosphate, pH 5.85 followed by 19 B.V. of 17.5 mM sodium phosphate, pH 6.3, respectively. The results are shown in FIG. 20.

This biphasic elution procedure using 6.5 B.V. of strong elution solution generated an RP-HPLC analysis profile for isoforms 1 and 4 that was similar to the profile obtained with 17.5 mM sodium phosphate, pH 5.85 alone (FIG. 20, panels A and C). Although the IFN purity (>96%) and poolable yield were improved over the standard procedure, a low amount of isoform 4 eluted in the latter fractions of the IFN peak. Shortening the strong elution step to 5 B.V. did not improve isoform resolution; instead more isoform 4 eluted during the latter fractions of the isoform 1 peak (FIG. 20, panel B). 2. First phase elution with 17.5 rnM phosphate, pH 5.85; second phase elution with 17.5 mM phosphate, pH 7.9.

Since the high pH phosphate buffer (pH 6.3) did not overcome isoform 4 eluting in the latter part of the IFN peak, a still higher pH was tried for the weak elution step. For this experiment, IFN-a2b preparation 5 was used. The loaded and washed column was treated with 6.5 B.V. of 17.5 mM sodium phosphate, pH 5.85 and then 15 B.V. of 17.5 mM sodium phosphate, pH 7.9. The results are shown in FIG. 21.

Isoform 1 eluted as a sharp peak in a small volume, but surprisingly, a large amount of isoform 4 also eluted in this peak (FIG. 21, panel A). The resolution of isoforms A and was even worse when only 3.7 B.V. of strong elution solution was used (FIG. 21, panel B), contrary to our assumption that use of less volume of the strong elution buffer at pH 5.85 would abate isoform 4 elution. 3. A critical role for low conductivity in suppressing isoform 4 elution

The fact that elution with the 17.5 mM sodium phosphate buffer at either pH 6.3 or pH 7.9 did not reduce but instead increased the isoform 4 elution was perplexing since the high pH conditions should otherwise facilitate isoform 4 binding to the DEAF resin. FIG. 22 illustrates the overlay of the conductivity, pH and OD280 of several biophasic elution experiments. IFN eluted in a similar pH range in these experiments, but conductivity under the isoform 1 peak displayed different profiles. In all cases, conductivity during elution first declined, leveled off and then increased, forming a cup shape within or around the isoform 1 peak. However, the bottom size of the cup varied with the bed volumes of the strong elution. The cup became deeper and larger as the length of the first elution step increased, especially when pH 7.9 buffer was used for the second elution step. More importantly, conductivity elevated within the second half peak of isoform 1, especially after a short first elution step (FIG. 22, panels A-D, and E). The increase in the conductivity during the weak elution step correlated well with the amount of isoform 4 that co-eluted with isoform 1. This suggests that conductivity played a critical role in iso4 elution. High pH buffer, in particular, the pH 7.9 buffer, significantly increased the conductivity of the effluent, and thus enhanced the rate of isoform 4 elution.

D. Use of different phosphate concentrations at different pH values.

As higher conductivities likely accounted for isoform 4 elution, we performed biphasic elution on IFN-a2b preparation 5 using various volumes (2 to 6 B.V.) of strong elution solution (17.5 mM sodium phosphate, pH 5.85) followed by a second phase elution with 15 B.V. of a much lower phosphate concentration (5 mM sodium phosphate) at pH 7.9 to test the possibility that this elution would suppress isoform 4 elution. As expected, a much lower conductivity (0.73 mS/cm) was detected during the second elution phase under these conditions (data not shown). The elution results are shown in FIG. 23.

A relative small amount of isoform 4 eluted while isoform 1 was effectively eluted as a sharp peak. The isoform separation efficiency was greatly improved over biphasic elution using higher concentration phosphate (17.5 mM sodium phosphate, pH 7.9) for the weak elution phase (compare FIGS. 20 and 21 with FIG. 23). This further demonstrates the importance of low conductivity in suppression of isoform 4 elution.

The length of the first phase elution step using strong buffer did not dramatically change the elution profile for isoforms 1 and 4, except that increasing the length of the first phase elution step appeared to reduce the retention time of both isoforms on the column (FIG. 23) and there was also a higher amount (12%) of uneluted isoform 1 remaining on the column when a shorter length (3 B.V.) of the strong buffer elution was employed. The eluted isoform 1 peak was sharper and there was only 3% uneluted isoform 1 when the strong elution was performed with 6 B.V. of buffer.

Taken together, the biphasic elution procedure with 17.5 mM phosphate, pH 5.85 and 5 mM phosphate, pH 7.9 provided significant improvement over the standard DEAE chromatography procedure. However, one or two of the latest fractions in the isoform 1 peak still contained a higher percentage of isoform 4 than would be acceptable to allow those fractions to be pooled with the earlier fractions.

E. Effects of additional parameters on biphasic elution using 17 .5 mM and 5 mM phosphate, pH 5.85

Several additional parameters were examined that might affect the column performance, including the amount of IFN, second buffer concentration, column length, and flow rates.

### 1. Loading

Effects of the IFN concentration loaded onto the column were examined by varying the feed amounts from 0.5X, 1X, and 1.5X of the standard loading (approximately 3.5 mg/ml), which is 75% of the column volume. First phase elution was performed with 6 B.V. of 17.5 mM sodium phosphate, pH 5.85 followed by second phase elution with 15 B.V. of 5.0 mM sodium phosphate, pH 5,85. The results are shown in FIG. 24.

IFN loads at 0.5-1.5X of the regular loading gave similar elution profiles, yielding similar separation efficiencies of isoforms 1 and 4. As expected, the peak height was lower at 0.5X loading than the regular loading (1 X). However, the peak height at 1.5X loading was not higher but broader than that at 1X loading. Additionally a peak of OD320 (approximately 2% of the 0D280 peak) eluted at the 1.5X loading that was not observed at 1X loading. This indicates that some precipitation occurred during the 1.5X elution. RP-HPLC analysis indicated that isoform 4 was low across the isoform 1 peak under all these loading conditions although the percent of isoform 4 was slightly higher (1-2%) in the latter fractions of the isoform 1 peak at the high loading (1.5X) than at the lower loading (<0.6% isoform 4 in 0.5X and 1X). These data suggest that the column performance is very consistent within 0.5-1.5X range of the feed loading.

2. Phosphate concentration in the weak elution buffer

Different concentrations of phosphate in the weak elution buffer were examined to further examine isoform 1 elution following the first phase elution step and the results are shown in FIG. 25. Interestingly, phosphate concentrations ranging from 12.5 to 5 mM at pH 5.85 were able to inhibit isoform 4 elution from the column. Most of isoform 1 fractions contained less than 0.5% isoform 4. However, the peak height for isoform 1 decreased from 2.5 to approximately 0.8 mg/ml as the concentration of the elution buffer increased from 5 to 12.5 mM. Therefore, the weak elution buffer concentrations within the tested range (5-12.5 mM) affected the focusing or sharpness of the isoform 1 peak but were still sufficiently low in conductivity to suppress isoform 4 elution.

3. Column length

Biophasic elution on DEAE chromatography was run on 0.5 cm diameter columns of three different lengths (5, 10, and 20 cm) and on a 1 cm diameter x 29 cm length column. The results are shown in FIG. 26.

Clearly the length of the column had some effect on the isoform 4 elution. Although the sharpness of the isoform 1 peak displayed similar profiles with these columns, the separation efficiency of isoforms 1 and 4 differed with column lengths. As the length decreased, the amount of isoform 4 that eluted in the isoform 1 late fractions increased significantly from <0.5% with 20 cm length to approximately 2.5% with 5 or 10 cm length column. The larger column (1.0 cm x 29 cm) showed highly effective separation of isoforms 1 and 4 with all isoform 1 peak fractions containing less than 0.5% isoform 4. In addition, the concentration of the isoform 1 peak fractions reached as high as 3.6 mg/ml. The concentration was sufficiently high that some precipitation of IFN occurred even at 4 ° C.; this precipitation however could be dissolved by adjusting the pH or salt concentration as stated previously. These results suggest that a column length of approximately 20 cm is required for satisfactory performance.

4. Flow rate

The impact of the flow rate on the biphasic elution using first phase elution with 17.5 mM phosphate and second phase elution with 5 mM phosphate, pH 5.85 was examined by running the column (0.5 x 10 cm) at 5 cm/min or 0.5 cm/min. The results showed the flow rate did not significantly alter the prospective of the separation under these conditions (FIG. 27). However, running at lower flow rates appeared to improve the separation efficiency. This is consistent with the general observation that the lower flow rate improves the column performance.

### Conclusions

Based on our data in the previous report, a two-step or “biphasic” elution procedure for IFN DEAE chromatofocusing was developed in this study, allowing the effective separation of isoform 1 from isoform 4 in a small elution volume. Use of high and low concentrations of phosphate buffer at pH 6, or low and high pH buffer at different concentrations resulted in significant improvement over the single step elution used in the standard DEAE chromatography procedure, although some very late fractions in the isoform 1 peak contained relatively high percentages of iso4. The combination of a first elution phase with 17.5 mM phosphate at pH 5.85 followed by a second elution phase with 5 mM phosphate at pH 5.85 yielded the most satisfactory results in terms of the final isoform 1 purity, recovery, and pool volume.

The conductivity generated during elution played a critical role in isoform elution. Conductivity above the threshold value of 1 mS/cm appeared to enhance isoform 4 elution.

The biphasic elution procedure gave reasonably consistent results within the tested range of IFN loading (0.5-1.X of current procedure), flow rates (0.5-5 cm/min) and the column lengths (10-30 cm). Overloading the column increased the isoform 4 elution in the later fractions of the isoform 1 peak. The column length had an impact on the column performance, with columns of shorter lengths (<10 cm) producing less efficient separation of isoforms 1 and 4.

***************************

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

Patents, patent applications, publications, product descriptions, and protocols are cited throughout this application, the disclosures of which are incorporated herein by reference in their entireties for all purposes.

1-29. (canceled)

30. A method of separating the oxidized monomeric isofonn of an interferon (IFN) from one or more undesired isoforms of that IFN in a mixture of recombinantly produced isoforms of the IFN, the method comprising:
(a) providing the mixture of 1FN isoforms in a first buffer solution;
(b) providing a chromatography column that is greater than 15 cm in length and packed with an anion exchange resin that is equilibrated with the first or a second buffer solution;
(c) loading the buffered IFN solution onto the anion exchange column;
(d) washing the loaded column with a wash solution;
(e) applying to the washed column a strong elution solution in an amount that is 1 to 10 bed volumes of the column, wherein the strong elution solution comprises a first phosphate concentration of 10 to 30 mM and has a pH of 5.4 to 6.6;
(f) applying to the column from step (e) a weak elution solution in an amount that is 2 to 20 bed volumes of the column, wherein the weak elution solution comprises a second phosphate concentration that is less than the first phosphate concentration and has a pH of 5.4 to 6.6; and
(g) collecting a plurality of eluate fractions that contain the oxidized IFN monomeric isoform.

31. The method of claim 30, wherein the anion exchange resin is a diethylaminoethyl anion exchange resin.

32. The method of claim 31, wherein the anion exchange resin is DEAE Sepharose Fast Flow.

33. The method of any of claims 32, wherein the IFN is a Type I IFN.

34. The method of claim 33, wherein the IFN is an interferon alpha (IFN-α).

35. The method of claim 34, wherein the IFN is an IFN-α2 and each of the first and second buffer solutions consists essentially of 10 mM Tris, 0-40 mM NaCI and has a pH of 7.0 to 8.5.

36. The method of claim 35, wherein the first buffer solution consists essentially of 10 mM Tris, 40 mM NaC1 and has a pH of 8.0, the second buffer solution consists essentially of 10 mM Tris and has a pH of 8.0, the wash solution consists essentially of 10 mM Tris and 14 mM NaC1 and has a pH of 8.0, the first phosphate concentration is about 17.5 mM and the second phosphate concentration is about 5 mM to about 7 mM, and each of the strong and weak elution solutions has a pH of 5.85.

37. The method of claim 36, wherein the IFN is an IFN-α2, the strong elution solution consists essentially of 17.5 mM sodium phosphate and the weak elution solution consists essentially of 5 mM sodium phosphate

38. The method of claim 30, wherein each of steps (c), (d), (e) and (f) is performed at a flow rate of 0.5 to 2.5 cm/min.

39. The method of claim 30, wherein each of steps (c) and (d) are performed at a flow rate of 2 cm/min and each of steps (e) and (0 are performed at a flow rate of 1 cm/min, the IFN is IFN-α2b, and the concentration of IFN-α2b isoform 1 in the IFN solution is between about 1.75 mg/ml and about 5.25 mg/ml.

40. The method of claim 39, wherein the concentration of IFN-α2b isoform 1 in the IFN solution is about 3.5 mg/ml, the amount of the strong elution solution applied in step (e) is 6 bed volumes and the amount of the weak solution applied in step (1) is 15 bed volumes.

41. The method of claim 30, wherein the volume of each of the eluate fractions collected in step (g) is about 20% of the bed volume.

42. The method of claim 30, wherein the IFN is IFN-α2b produced in a recombinant bacteria and from about 3% to about 20% of the isoforms in the IFN solution comprise IFN-α2b isoform 4.

43. A method of separating isoform 1 of interferon alpha-2b (IFN-α2b) from isoform 4 of IFN-α2b in a mixture of recombinantly produced isoforms of IFN-α2b, the method comprising:
(a) providing a diethylaminoethyl (DEAE) anion exchange chromatography column that is at least about 20 cm in length and equilibrated with a buffer solution which consists essentially of 10 mM Tris and a pH of 8.0;
(b) loading the IFN-α2b mixture onto the DEAE column in a loading buffer that consists essentially of 10 mM Tris, 40 mM NaCI, and has a pH of from 7.5 to 8.0, wherein the IFN-α2b mixture is loaded at a flow rate of 2 cm per minute;
(c) washing the loaded column with 3 bed volumes of a wash solution at a flow rate of 2 cm per minute, wherein the wash solution consists essentially of 10 mM Tris HCI and 13 mM NaCI, and has a pH of 8.0;
(d) applying to the washed column 6 bed volumes of a strong elution solution at a flow rate of 1 cm per minute, wherein the strong elution solution consists essentially of 17.5 mM sodium phosphate and has a pH of 5.85;
(e) applying to the column from step (d) 15 bed volumes of a weak elution solution at a flow rate of 1 cm per minute, wherein the weak elution solution consists essentially of 5 mM sodium phosphate and has a pH of 5.85; and
(f) collecting a plurality of eluate fractions that contain isoform 1.

44. The method of claim 43, further comprising combining the collected eluate fractions in which the amount of isoform 4 is less than a desired purity criteria.

45. The method of claim 44, wherein the volume of each of the fractions collected in step (f) is about 20% of the bed volume.

46. A method of separating a desired isoform of an interferon (IFN) from one or more undesired isoforms of that IFN in a mixture of recombinantly produced isoforms of the IFN, the method comprising:
(a) providing a chromatography column that is at least about 20 cm in length and packed with a diethylaminoethyl anion exchange resin that is equilibrated with a buffer solution, wherein the buffer solution consists essentially of about 10 mM Tris and a pH of about 8.0;
(b) applying the IFN isoform mixture to the anion exchange column in a loading solution of about 10 mM Tris and about 40 mM NaCl and which has a pH of about 8.0;
(c) washing the column from step (b) with about 3 bed volumes of a wash solution, wherein the wash solution consists essentially of about 10 mM Tris HC1 and about 13 mM NaCl, and has a pH of about 8.0;
(d) applying to the washed column about 6 bed volumes of a strong elution solution at a flow rate of 0.5 to 2.5 cm/min, wherein the strong elution solution has a first phosphate concentration of 15 to 25 mM and a pH of between 5.7 and 6.1;
(e) applying to the column from step (d) about 15 bed volumes of a weak elution solution at a flow rate of 0.5 to 2.5 cm/min, wherein the weak elution solution has a second phosphate concentration that is less than the first phosphate concentration and has a pH of between 5.7 and 6.1; and
(f) collecting a plurality of eluate fractions that contain the desired IFN isoform; and
(g) combining the collected eluate fractions in which the amount of the undesired IFN isoforms is less than a desired purity criteria.

47. The method of claim 46, wherein the desired isoform is the oxidized monomeric isoform of the IFN and wherein each of steps (b) and (c) are performed at a flow rate of 2 cm per minute.

48. The method of claim 47, wherein each of steps (d) and (e) are performed at a flow rate of 1 cm/min, the IFN is an IFN-α2, the concentration of IFN-α2 in the ITN solution is between about 1.75 mg/ml and about 5.25 mg/ml, the strong elution solution consists essentially of 17 mM sodium phosphate and has a pH of 5.85, and the weak elution solution consists essentially of 5 mM sodium phosphate and has a pH of 5.85.

49. The method of claim 48, wherein the IFN is IFN-α2b produced in a recombinant bacteria.
